SEQUENCE LISTING (29 sequences)

SEQ ID NO: 1 - DNA, 4834 bp, Toxoplasma gondii
cctagttgtgttcgcaacagtacaccgtcctgagtgagtcgagaacatcagagatgagcacacgcaatagcggtccgccaagggtgcatttgtccacatcgggatgcacagagtggcacgagtcgcacaaaagcagatactagagacaaggagagagtgcggcctaaccagaattcgactcagtttcttgacccattcgttagggtcggtctcagcctccttcaggatttccgtcaagacatctttgctagcttcccgctgcagacatgaaaggcagtgtcacgcataaagagccgattgaaacgcagtcacagagatacgaagaaatcaaagcccgtggaaagcgaacggctgggatgtagctgagaaagcaaattcactggcggtgcaaagagccaatgaaatcagggtcgcgtagaggaactataaaacgtgaaaaacgtgccttccgagtctcgcaaaggtgcgcatcgatcccacatttgagagaaggttgcgaggcagtaataagggcaggggagaggataaaatccgatagacccagttcttggtctcccagaacggggacaggaccggacgcctgcaagggtggatcacaactccagaggcaaagccgccacggaggaacggaatccatgaccgagtggaattataacgaagaggtgtttgtcgtcggaatggtgccaagacacaaaaaaagaaatgtttagacgctcgactgtgcactagcggggggcggggtgcaaaagggacgagtgtgctcagtggttcggaggtaactgaaaaaacggtgcaaaatatggagccttacgtggagccgcagggggcagaacagatgtctcagaagaaagtccgagagaacagaagaaaaacgagaaaagtgatgggcgactcatgcagagtggcgcgacgagtctgtctctcagacgagcttaccagtgctgggcggaggtaaaggaaagaagtcaagacgcggaccttgaggggggtggacagcatgatgaatcgctgatgtatgtactttagaagcgcaggagttaagagtcgagcggcatggcaggacgaccagttgtcctttatgcttcgcagataggcaatatatctgctgctgagggcctcatttctggagagttgcgttgtccctgtcgtcgcctcatcctttatctccgtgtttgtctcttccagggcagccttctgactaccgcccaacgggcttccttcttcggattccatttgagatagccgtagaagcagaggaagagccgtcagaacgcttgccgcggcagaaaaacacttaaagggcgtcacaagattgatataggcaagaggaatggacgtcaacaggctgattcataagtgacgctcccagtaagtggcggacagccatgaaaatgagcggccgagtttgcagaaacagagaaagaggtctgcatcctggcgaagagccgccggacaccctgcttctctttcacagttcgtaggtgccaagaccaggaccaaattatcgcccttcttagcaaaccttgagccgagttaccggagaggttagccgaaaaagaatcgaaacgaagacgccattttttgtctccattgcacacggacggaccgtagcttgtctctcagcatatcttacgacgttttgcggctgttatcgctaacacaccacaaagagaaatggtttatcgaaaaacttgttagccggatggtaaagagatgcagaaggcagtccgcagtaattcggttttcgtcagttgtggcgtgctggcacactcacgtttttccagcgtcacatgctgcctgattcacgcagaaactgcatgtgcgctgcgtgtctcgcctgcctcaggatgcccttgtcggccgatagtgaggaaggaaaaacggctccagcaaaatgttggttctattcggcgagtgccggtattccttccacaaggtcgagacaccgtcgagtgttttccttccggactgaaccccggaaaagtcactttgcaccgtagattccacgtgctccagcgcggctgtcaattttcgacactgcgcgaacggcttgccaacaagaccaggctcgcgcgcccggcttttcacattcccgacggcttatatacggaaggctttgccaggcgtattctggccgcgtggggtcgaaagaaagtcgaaaaagagcatgcttgtcaagtgcatgcggccatgtaggttgctaggacccctgttaaatttccagggtgcggggcaactaagtggcctctcttcgcgtcgtcttcggactgttctctgggttggcctcgcttcgccacagacacttgtcgacgcgtctcagggagtctgagcccgttgtatttttttcgctgtctttttggcggttcccgtttcccctcgactgccgactctcccctctcccgctccgtcgccaccatgaagtctgtttatgcctgtgagactatcaccatccctgcgggaggtaagtttctcgacctacgagagggtgaactgcggagaagacgaatgaaacattgccccgcttgatctttgagggagagttgccagattctgcggctccacagccctcgttttttttcctcccgcatgtgttagatgtgtcccgaccccgagggaagcgatcgacacgctgggaaggaacgcccgatgagcggaaaagttttggaattcaggccccgatgcgcaaagtggcaagtgtcttggaccccactgaggaagccgaacagcagcattttacagatcttcgccactgaggaggggggcggatctgggaggtgaagaggcgcggaaccgtgttccacttggcttttctccgcattcgctgtgtctgctctgcgtcaaaaatccgcatgctttgttgtcattcaaagaggtcatctgggcgccttgttctttgttctgccgcatccacaacagtctgacccgccagagaatacggtctgttctgtccggtgactggcgatggggaaatgggggaaactgtgtcgtcagcgagtgaaggcgttttttagtggaatttctacattgtgcaagcacacagaaggtgtcccgtgctaatatctggaacagtagattatgattaggtagtggaacagggagagcgtctgttgtacatcactgtctgcactcgtttgtactacaacgaagttgttgatgcgctgacttgggtgtcgattgcatagacatagcgtggaaaagtagaagacagggttgtatgcgaggctctgtgtgcacctgtttcatgtggacaagaccaccgggcatatgctggctgttgcttcaacacgctgccgaaacatgtcacggcgttgcgggggaaaggagtcgttgtagaaaccatagagagagttgaggtagctcttgatgtctcgcaaaaatgggactggcacctgttgtctgtgtcttcgattaacacgagcgccgccactgcgtttgatgctcgctaactgggcagcgtcgtgtacgtacagctcgaatagcgtaattgtgtgtttttgtactctttctgtttgagtttcatcaaagagggccagccacaaaatgggagcagggggatattggagggcatatgataagtgccgcctgtgtgcatacttctagaatagacaggaattcgagagcgaagctgtctgaacagagatctgcaggttctggtttgactgtgtaggcaggtttctgtagcgacgggagtcgcaatgcagagtgccgcttggtattggttgtttcaagatgtttgcatccctctgagagcaatcgctttttgtcctgttttgcgtgtctctcggctgtgtgccttctgaaagaaaatgttgcatccgtttgcggttttctgctgcagtcacggtggatgtgaagtcgcgggtggtgactgtgaagggcaagtaggcgaaatcacgcgtgcattccgccacctccctgtcgacatccagaagaccaagtctggaaaccgactgaaggtcgagatgtggtatggaacctgcacagacctcagctgcatccgcacgctgtgctctcacatcaagaacatgttcactggtgtgatgaagaagttccagtacaagatgcgcttcgtgtatgcacattttcccatcaacgtgaacatcagcggcaacggaactgtcgtcgaaatccgcaacttcttgggcgaggagcgtgtgcggatcgtcaagatgcttccgggagttaagtgcgagaaggccacaaacgtcaaggatgaaatcgcgctcactggaactgacgtcgagctcgtctctcgatcaggtagaggctccgaggaactgaaaaggggcgtggtgtgccggtatgcgcgcatctaatatgagttttggaggtgcggaggcagcaaggaagcgtatagatgtgggcatttatgaatgtgcatctatgttggtgtaattctgtgtatgcctgattgcgacgtgcccacaaccacctccaggttggaagaagagagaaactgataacggtggaccccgagagcgggattaccgggaactctcggacggtcgttgtatactcatctcacgtgggcgagggggaggtggtttgtccttcgatgttgccacagatttggaggtgaggtgtcttcatatcctgcatgtgtgtctgcaccagcagatatgttaactgccaggtgagacacgttgtcgagccacaggtatttttgtgtatctgcattgcattaacatggtttgtattcttctgttctgtatgcttctcttcttcagcggctctcatccatcaatcgactttggtcaggagaaaggatatccgtaaatttttggatggcatctacgtgtcagagaccagcactgtcgaacaggacgcgtaaatg

SEQ ID NO: 2 - DNA, 4338 bp, Toxoplasma gondii
  misc_feature (565)..(566): n is a, c, g, or t
atcgcatgacctgatcacgcacggaaagaaacgaatagtcgccatttgaagtgagatccgtgtgaacaggtcagatcaccgaatcggacgatatatgcactgaaggcagcggagccagctgtaaacaaagaactggacagcagctaccgtagctgtagacggacgcgacttcgagagcgtccacgtcaaacctcaccgatctcgccctcataaagaggcatgtgggctgggagatacaggggtgaagaaggagagacaatttgcgtaaggaggcgaagctttcgatttccaggtgcgattggagtcgccgccacaggagacgcgaactcctcaaaaacggacacggagaagccctgtgcagagacaacggaaagaatgtcctgacgagagagttgcaaaagaatgttgaacaattaaagcaatgatgcagactcgaagatctaacgcctcgcaggtctcaacggttgctgtgatcgcccatttacacagtccttaagttgagtgcatgagaggctttgcagctcaaggcaacgctgtaaacagcagtgttatgaatcggttgccnntattgaggcgtctgcgtctggtcggtccatcaagccaaaagacgcttgtaaacaggatnntccattcgaatgnngacagacagtttggcaactgtcatcacacgtgacgttaaaaggcaccgttaagcgcatgacaaggaaaggtcacccgcgatttacacacaccaggtgccgtagctgtcgatgaatgcgaattccagagtttttctctccgacactacataagctgtaaatgctcattctgtcattcgttgaccgtgtttactacggggaatcgagaaacggaatatcaagaacacaggctgtcaaaagacaccgcgaaacctgcttgcggaatctaacggttgcctctggccatttatgtgtttctcgcctgtgccttgttcgctgcagacacagcctgagtccgcagcgaggtaaatacgaagaaaaacctgacgagctctgtcagatctgtacaagcgacagaagcggattgacagaggagagtgcgcgacggtgacgagagtgagagtcgactacgaagttagaggacaccagggtggcgaatgtgccaatacgcagcttgaaagggtcgagatcgacaatcgaaactcacttcactcgttaaacaatcgagcgtttgctgcaggttttgtttggggcaccccgcctttgccttcccacccatcggattcagcccgcagtactccaccagcaaaacagcatcgaggccgtatgcctcgaagaagtctccaacctgcaaaagaaaggcaccacgtcagagcaaggaaatcaagactcaaccaggtgtacagacaccgcataccgtcgccaggaaccccggtctaggacaactttgctagtgctgctcaaaaggtggaacggagaaggcgagacagcagactggcggtgccagttcaaatcaccactgcctgaagcgcggggaccgagacagtttgcgatgttggaattcctgtgacgcacacactttggaacttgcctgaataatcagaactttgtcgggccgaagtcgtttttgttctcgtacgaagacgaggagaggaggcatatgcagggagtcaggaaccatcgacggatggtcgaaggaaagaaaagagagctgccgccgggaagcgggacgggaaagaagcggcagccttgccaagaacgtcgggtgtactgccaccgagggaggcgggcagctttctgcagacgaacgcagaacggagcagttttcttccgctcttcgactcggcttcggtcccagcaggttgtcgcgcgtcggcgcttccggacgcttctgcgtgtggaagaagcggccggacaggcgggatgcgtttgaagggaaatgagtctgcctgtctgaaatcgcgtgaggcaactgaatggtcggacgtgcgaggcgtcggtccggatggacaacaaaagcgacgcgtcgagaagaggcaagaaagggcaagctggcgcgacagccacgcaactggcctgctgcgtcccttgcagctgccgaaaagaagtccagcagctgatgctgcccaagagtggcccaccctccgcagctgctgtcgcgtttcgcgaccttctgtaaaaaactcttggacgtgcggccgtgcatcttgttcgcggaaagcaaggcattgatcgaccagtacgcttgcggaacggagcaggctaaaccctgccggcaaccgcgctgtcgcggaaacatttccatcagcagtctcgcgttgacctccactgagatgcacacaagtgaaaagagaatcgtctgcagtttccagcgatagcgcgcgaaagaagcggagccgcggagaggcgcccccggacccgaagcgggccgcagagcgcgagaggcaatggagcaaaaaaattcctgcctagaggagaatcgccattcctggtcacggtcactgcatacagaatcccctcccatcgtgggtctgatgaaaaagagaaaaggacaccattgtggtgtggagccgcggtgccgtctcggctttcccgttggtcaatgcagaagcgcgtccccagtgactagagccgaaccaccggcgactccaataaaggggccttctactccattcaggggtgtcgcatggtaaaactgagttctctaatccatggcacacctcggaaaaacactactcacagtcgtggaatatcttaaatagtcgagcgctccagagtaccaagctgcaatgcccagctgcctctcggacaacaccggtggactaggcagcgcatcaaggagacaccccccagaggcggccttactccagaacatgcgaaggatgtcactgttgatgcaaaatcagggtactgaccatcaagtgaacagtttctagagtatccacgtggctccaaaaagtaaagtatggccccacatcaaaccgagacyagcctgtcttcgttagaaacgctcgtgccgatgacgtcagtgcatgcacgctgcagacacgacccaaagtagctgatacggtgaggaatgagcatgctgccgattcaaaatcgtcagtcgcggctacagcagtgttttatgtacatcctcgttttctttttattcaagaaacccaggtaacatgtgcttgaagggcggacttgggtagaatggccctcgaacgttggtgccgaggggttcggtgcagccttccatcctgggctgtaatgtgtcttgcgtattttctctgcgggcctgcagtcggaactgccttcatagtccctcaacgcaacctgcatagctttaccaggaatttccgtgtccttcccttggttgggaagcgatggtttgcaggagatgcagctgtgcaacactgtaaaggctggtttcagcaaacgggactctcccctcggtcactcccagggagtgtcggccaggtgacggcctggtctgcggtcacagaaggatgtcctttcgaagatgcagtccatgaaacttcgcttcctagtgactgggtggagaggagtgaacccacagctctagggacggatgcgacactctcaagcattccatccgagctacctgagcctggaacgccgcggaacagcttctcatggggcacccggaaaaggtactgacgagagaatgctttctcagaattgatccttcgccttctgtgagaaacactgtcacgaggactgcaatcttttcaggtgtatcttttctgtacagctgtgcagcgttcacgcttttgaccggagagtacccgtgatgtgacttgcgggttgggtagagcctagtccagctgtctgtcatcttctgaacgggagttaggaagccgtaacaactaatatgtgcatcgcggtttctccggtgtgcgcgtgggcaggtcttatgcaagggttcggcttccccctggttctggaaagctgacgatcaacaacagagacgcagccgactatcttcaagacaatccatggtggattcataattgcatcgcgccactcatggaactgcagctggagaatgaatttgatatcattgcggaggtaagccacgttagctagcttcgggtgacagtcgatatgtttcgcaatgatttcttacagcgtctttcctacgtttttgcttgccttcgacatcaggcccacgggggaggcctcggcgggcagtctggagcaatcatgcttgctgtcgctcgggagattgtgcgacagcgaccggaactgcgaccccctcttcggcgagcaggttttctgaccgtagacgcaaggaaggtcgagaggaagaaatttggtcttcggaaggcgagaaagaaggaacagtacagcaaacggtagggtgcgtaggtca

SEQ ID NO: 3 - DNA, 3639 bp, Toxoplasma gondii
cctcgcagagattgtcagtgcatgacacaaccgcgaaaagccggcagccgcggtaatacggggacgaggaaaacgactgagcgtcacaacagaagcagccgagtaaacggcgaaggaaatggaaaggacccaagtaaaatttcttgaagaatttcagcgcaacaactctgcgggttcttgcgaatagaggaatttcacttcctcatcgtctgatttatgctttcatcatctgccgctcaacagccgaataaacggttctcggtcgcttccttaaactctacttcagtagttgaaactcttttgcttcacgagccttcgtctcagccctcaccgtcctgagttctgtctttgttgaggaaagctcccgctgaaaaaacaggactttgtttgcagattttcatgtgtactggaaagtgagatgtgacttggggaagtccgctttaaaatttccattgttttctcaaaatgaaaagtctaaaaaatcgaagtgcgtgccccgcgaggaattcccctctgcagatttgttttgcatttatatgtcgtttttacggagaaaagtcccaagctgctgctccttctctaactagatgttgaacgctagcacatatgcaccagatgcttctgaagtatacctaaacgcaccttgggaacaactgtgctcccattcataaaactcatacaagtcaccaagcatgccatacccgtgagacataacaacggaagctagactactcccccctgttattgcacactatcgaaaaggattcctaggtttctatcctctgccttttcctggggcacactgcagagaaactaccgtgcgcgctacctcccgacgtgcgaggcgatagcaaaacgcttttgaaggaaaaagtcgagaaatcgacgactgcgtctcttgaatccgagagagggatccaacccaccgagttctctgcatgtgcagcatctgcaagaacgtgataatgcatgaactcgatcatcgccttatctgtgtgcatgcattttcgaaaaagaaaggcgttttctgcgcggagactcgcgcggaggcaagacgagactttctcctcttccaaactgcgagccacgggggcgcatgcaatttgaacatcacgcaaaatcccaaaacgggtggggtggagccgcaaacttttttggcatgcagcgttgagcctgagctgcggtgggggcttttgtcgcgagcgtggggtgccgcgagagagcaacgcggcgctacgcggccgacgggtcttctgggaagcctcgcatttcctcgacgggttctcccctcaattctcttcctttctctgcgtcttcctcaggtggcttcgtcaccggtttttctcctcgcgttcgtgctccgctgtgtgtccggagtgccgcgacagatcgagggcgttctccgctcccaccttgcggttcccaatttcgatttttctccgtcaccatggggcgcatgtacggtcctggaaagggcatgtctgccagcgctctcccctggcgcagaaagcccccgacatggctgaaaatcaagccgtcggacgtcgaagagcacattgccaagcttgcaaagaagggccagaccccctcccaggtactttcggcgggaagaagggagaaaaacgacggagttgccgcggctgcggtctggggaacaacggggaagtgcacaggaaaaatacgcgttctccaggtcatcgggggaaaacgctcgcgaatcccagcccctcgactctccgcagttgtttttcgcagtcttttcgccctcggaccgttcagcggagatggggacgagaatcctctccctccctctgctgagttttcccgcctctctcgtgtctcaaaaaaggctgcagaaatgctcgcttgcctcacagccggacctctctgttgaccaaagcgcccagtcgggatttcttgcgcggggatgggctgaagcaacgaaacgggatggacgtttgtcggtttccccgctgtcgtgtgacttgctcgaggaaccaaagaacgggaacgagcgggaggaagccgggggaaaatttgcgttcctccccgcaaaacctatccgcaaaaatgcctcgtttcgcgaactgtggacgggggggaacaacgcgttgtgttgtttgtgcatacctgactgacacggtcctgcgcgtgggtggctgggatccgagcggtccgaagacagtctctcggaaattcgcgagcggacctactgttgttcactgaacggttcgcttctcctgtgaaatcaacatggtttcttgtgcagtttaccgaaagtgaggacgacatgttttttgtgaccgtggcgcggccgttccgcgtcggcggcaaaccggacgatccaatgcggcgaaaccgggggagagtcattgcacacgatgaagtcgcgaacgaccaaggcaattttttcggaggtcaaatcacatctttcgagaagtatgaatgcatgtggaaggtcgtctgtttttccattctccacgttcttcttgcttttggctgcttgtgcttctctggcgacttacagatcggcgtcacactgagagactccttcggagtgccgcaggttaaatccgtcactggcaacaagatcctccgtatcctcaagctccaaggtaggtttacgcgttaagggaaacactagtttcaatcttctcgagaacactggaggggcggagatcggggcgcagctccttccaggctcaagaaggtctgggagtgaaaaacgaacgcaaatgcatggacgttgtatatatgtatgcctggatacggtgtgagggtagcgcctttggcaggagcaagtgtgaagtttgcgtctgtttggagaaggaatgacgccgcgtcgtcgcgagggcgttctctgccttcccggttctctgttctttgagaaagaacgtttttcgcgtttctccgcagtgagaggttcccttcgaagaggcacctagatcagtcgactcgttcttgaggaggctggccttcgtcagtgtgtctgctgcttctctcactgcaacactgtctcccttgaagagatttagcgcagatgctgattttctggcgttcagctctctgccgtcgccctctccaacatgtcaagaagcacgttgcttgtctccctctttgtccagcaaagtggagtttttgtatgcgtgcaatctatgcaatcgagagcttgctgaagcgacgttgctctcctctctcccaagtgtatgctctccgcgtttcttcgtctggttaaaaagaaacggcgtcgctgtcctttccttcgtggcgaactcggtattgtttctcaaatccgatctactgtcagccgtccaagtgcctgtctgacctctttcctcgactcccgcatgcacatttagagcgcgtggaagcgactgttcaagtcctcctctcatctgtttctctaggttctgaagagcgccaagtgcgttttcgaggtcctccagacccggcgccaccagtgttctcccgactgttcttttttttcaggtcttgcccccgagctgcctgaggacttgtactacttgatcaagaaagccgtgagcgtgcgaaagcacttggagagaaacaggaaggacaaggacgccaagttccgtctgattcttgttgagtctcgaattcaccgtcttgctcgctaca

SEQ ID NO: 4 - DNA, 2748 bp, Toxoplasma gondii
ctgccgcttccttaacctctggaaggggttgaagctttttcgcactgaaacgcgagagacacgaatgagctgaccacttttcctgcattcgctggccctgtaccggccgcattctctacttcgtacaccttcactgtactcacacccgaaaacttcagaagtcgggctttgctgcaggcgactcagggcagaggagaaggtgaacatgccttccccaattttgcccgcagtttgcctcgggttgctctcttccccaacctagatgaaatgaaaccactcgatcagccatgtatttcccgcaaaaggttggctaccaagcccacatttggtaagcactctggaagatgcggcgccacggaagcaaccctcgaccgcaccgctgtcgcggggtctcgacgtcgtccgcgttaacatgatgtcccgccagagcttccactgttctgacgagtacatggatgcaataagcaagtcgtctccactcgtacacgaatgtccagcaccagcaagctgcatcacttcaatcgcttggtagcccaacgcttcttttgtctgctttctgtttttgctgcgagactgaccgagcacgaagcttacacgaaacgcgaagctctaatcgcctacatcccactccttcttcagaaatggcagggaagctacactcttgttaacgtctctaggtttaaagggtttgtgcaccgagccgatcacgttacggacacgggctcggggttcactacgttacaaggaataaacaactaacgcacaatcctggttacattcgggcccacagcatataacccctccggaggcttgtattccagctatcgaaaaaaaagcaattcgatgtaattccctccaatagccccgagcgyatgtcatctacaagtggcagccttggtagggacccctcttgggtatgcccggacagatgcgccagtgagatctttaacctccgcgtaaagataggtgtctgctgtagcggtgcgcttttttgtgttgcatgcatgcatcaagctggccgggacaaggcggttgccccgatggatggatggcaaaaccactgtgctgcaggcagcagccctccctcggaggctcctcttgtggggcacggcgcatgccccagcacaacgtagcggccttgtccagcatggacgaaaagcagtgggggagactcccagaggaaagcgttgccatgcaaaggggaaacaggggatttttgtcacgacgagctgtttgtccacttttgtgaggtggtaatttgacacagttctcatccctgttttgtccaagatggcgtcgaccggcaacgtaaggggattctccgcactgcattgctctcttgagggagaaaaggccgccgtgaaaacatgctgttctccaccagttggcagattgaagcagtctacgggaagatgcacggtttaatcattgttgaatttcgtctgcagtctgactcttccgtattggagcacgcgatgtctcgttgcgtgtgaaatcgtactgtctggatctctttgagtgaagaacacgcggcaggccgcagtttttttgcagggccttggtaccaaatgcttgtttacatatttgccttgtcgagttcttttgcatgcttttagatatgcgtggagactgttaaatcaacaaccgctcggagatattgtgcgcggcccagcaatgctctggttccactcccgtcgtgagcagggaacgcatggtgggcttttgtggcttctgtgtgtatgccgtctgcagacttgcaaaaagagaaagttcgtcaaggatggtgtcttccaggcggagctcaatgagttcctctcctgcacactgtccgaggatgggtactcgggagttgaagtccgtgtgactcccatccgcacagagatcatcatccgcgccaccaggactagggaagtgctcggcgacaagggaaggcgtatccgcgaattgacgtcggtcgttcagaagcgattcggcttcgcgcccgactcggttgagctcttcgccgagcgtgtggagaaccgtggtctgtgcgccatggcacaggcagaatcgttgcggtacaagcttctaaaggggcttgcagtcagacgcgcctgctatggtgtcctccgccacatcatggagtccggagctaaaggtgagtgctgacaaagtgccatgtattgtatgaggtaacttgaatttagagtgtgaacaaaaagcattagtcgactgtcacacgtatcttcgccggacttttttcttttcaggttgcgaggtcgtcgtgtccggtaaacttcgcgctcagcgtgccaagagcatgaagttcaaggatggttacctgatctctactggagagccctcgaagatgttcgtcgaccaagcaatccgctcggtgcaacttcgacaagtaagtttcaaattattaagcctcagttacgtagtaaagggcaatttgtgtaggagctagtatgtacagaggcagtgtatgtgtgttttttttgcagggtgttcttggtgttagagtcaagatcatgctgccgcatgacccggagggcaaacgtggccccgcgaacccgctgccggatactattatcgtgatggatcccaagccagagatccccgttgtgcagcctgaggagatggacgagggagtgctcggtccaatgtaatgagtgattcgtgcgtgactgttgatttatgggaggagggtgtccacatgtgcgtgaccgtggagcagccgcttaacgaaattcgcatgctccttcg

SEQ ID NO: 5 - DNA, 31 bp, Artificial
  primer SAG3-FW
cgataagcttcgaatctctgaacggatgtgt

SEQ ID NO: 6 - DNA, 33 bp, Artificial
  primer TUB5-RV
cgagatctgggaattcaagaaaaaatgccaacg

SEQ ID NO: 7 - DNA, 30 bp, Artificial
  primer TETAVR5-FW
cgatcctaggatgtctagattagataaaag

SEQ ID NO: 8 - DNA, 33 bp, Artificial
  primer TETPST3-RV
cgtctgcagttaagacccactttcacatttaag

SEQ ID NO: 9 - DNA, 21 bp, Artificial
  primer T3
attaaccctcactaaagggaa

SEQ ID NO: 10 - DNA, 31 bp, Artificial
  primer SAG1/1634-RV
cgataagctttcgggggggcaagaattgtgt

SEQ ID NO: 11 - DNA, 27 bp, Artificial
  primer REV 13A
gcgccccatggtgacggagaaaaatcg

SEQ ID NO: 12 - DNA, 27 bp, Artificial
  primer REV 13B (nested primer)
gggaaccgcaaggtgggagcggagaac

SEQ ID NO: 13 - DNA, 30 bp, Artificial
  primer S13PROMFUS FW
gcataagcttcctcgcagagattgtcagtg

SEQ ID NO: 14 - DNA, 31 bp, Artificial
  primer S13PROMFUS RV
gcattctagaggcagacatgccctttccagg

SEQ ID NO: 15 - DNA, 33 bp, Artificial
  primer LACZ-AVRII FW
cgatcctaggatgaccatgattacggattcact

SEQ ID NO: 16 - DNA, 31 bp, Artificial
  primer LACZ-PSTI RV
cgatctgcagttatttttgacaccagaccaa

SEQ ID NO: 17 - DNA, 50 bp, Artificial
  primer S13INSTETO+3FW
ggttctcccctcaatccctatcagtgatagagatctctcttcctttctct

SEQ ID NO: 18 - DNA, 50 bp, Artificial
  primer S13INSTETO+3RV
agagaaaggaagagagatctctatcactgatagggattgaggggagaacc

SEQ ID NO: 19 - DNA, 51 bp, Artificial
  primer S13SUBTETO-23FW
ctacgcggccgacggtccctatcagtgatagagatcttcctcgacgggttc

SEQ ID NO: 20 - DNA, 51 bp, Artificial
  primer S13SUBTETO-23RV
gaacccgtcgaggaagatctctatcactgatagggaccgtcggccgcgtag

SEQ ID NO: 21 - DNA, 32 bp, Artificial
  primer S13NOTI-FW
cgatgcggccgcgtcagtgcatgacacaaccg

SEQ ID NO: 22 - DNA, 32 bp, Artificial
  primer S13SACI-RV
gctagagctcctgtaagtcgccagagaagcac

SEQ ID NO: 23 - DNA, 23 bp, Artificial
  primer M13-REV
aacagctatgaccatgattacgc

SEQ ID NO: 24 - DNA, 20 bp, Artificial
  primer S13CL FW3
cgatagtgtgcaataacagg

SEQ ID NO: 25 - DNA, 21 bp, Artificial
  primer HRCHECK II 5 S13-FW
gtcgagtcctgtaggttcatc

SEQ ID NO: 26 - DNA, 21 bp, Artificial
  primer HRCHECK II S13-RV
ctccgaaggagtctctcagtg

SEQ ID NO: 27 - DNA, 17 bp, Artificial
  primer T7
aatacgactcactatag

SEQ ID NO: 28 - DNA, 32 bp, Artificial
  primer HXGPRT/BGLII-FW
cgatagatctaaaatggcgtccaaacccattg

SEQ ID NO: 29 - DNA, 31 bp, Artificial
  primer HXGPRT/PSTI-RV
cgatctgcagttacttctcgaactttttgcg

(19) United States
(12) Patent Application Publication
(10) Pub. No.: US 2005/0244437 A1
(43) Pub. Date: Nov. 3, 2005

(54) Live antenuated parasite vaccine

(76) Inventors: Nicole Francisca Johanna Van Poppel, Nijmegen (NL); Arnoldus Nicolaas Vermeulen, GZ Cuyk (NL); Theodorus Cornelis Schaap, Beugen (NL)

Correspondence Address: INTERVET U.S.;PATENT DEPARTMENT, PO BOX 318, MILLSBORO, DE, 19966-0318, US

(21) Appl. No.: 10/526,731

(22) PCT Filed: Sep. 19, 2003
(86) PCT No.: PCT/EP03/10696

§ 371 Date: Mar. 4, 2005

(30) Foreign Application Priority Data
Sep. 20, 2002 (EP) 02078953.3

(51) Int. Cl. C12N 1/10; A61K 39/002; A61K 39/005; A61K 39/008

(52) U.S. Cl. 424/269.1; 424/273.1; 424/270.1; 435/258.1; 435/258.2; 435/258.3

(57) ABSTRACT

The present invention relates inter alia to attenuated live parasites of the phylum Apicomplexa and the family of Trypanosomatdae and to the use of such attenuated live parasites in a vaccine and in the manufacturing of such a vaccine. Furthermore, the present invention relates to vaccines comprising such attenuated live parasites and to methods for the production of such vaccines. Finally, the invention relates to specific tet-repressor fusion proteins and to attenuated live parasites according to the invention comprising such tet-repressor fusion proteins.

The present invention relates to attenuated live parasites of the phylum Apicomplexa and the order of Kinetoplastida, to the use of such attenuated live parasites in a vaccine and in the manufacturing of such a vaccine, to vaccines comprising such attenuated live parasites, to methods for the production of such vaccines, to specific tet-repressor fusion proteins and to attenuated live parasites comprising such tet-repressor fusion proteins.

Within the regnum protozoa, the phylum of the Apicomplexa and the order of the Kinetoplastida, more specifically the family of Trypanosomatidae, are known to harbour several notoriously pathogenic parasites.

The family of Trypanosomatidae harbours i.a. parasites belonging to the genus Leishmania and Trypanosoma.

Leishmaniosis is a term for a variety of disease manifestations caused by Leishmania. The disease occurs most commonly in dogs and humans. The parasite is transmitted by sand flies to a mammalian host and is prevalent in all tropical and subtropical zones of the world. In the host parasites are taken up by macrophages where they stay and multiply, causing chronic inflammatory processes. Clinically, the disease in dogs is characterised by loss of weight, anaemia, pyrexia and lymphadenopathy. Cutaneous lesions are frequently observed. In humans multiple Leishmania species are infective, of which the most pathogenic is L. infantum, causing severe, visceral Leishmaniosis (known as Kala azar), which affects spleen, liver and bone marrow, and is fatal if left untreated. Other pathogenic Leishmania species are i.e. L. major and L. mexicana.

Multiple species of trypanosomes are known, causing a variety of different diseases in both man and animal. Two trypanosome species in particular, are known to be pathogenic: Trypanosoma brucei and Trypanosoma cruzi.

T. brucei species are present in African countries and cause sleeping sickness in humans and Nagana in animals (cattle, horses, pigs). T. brucei is transmitted by the TseTse fly, delivering the trypomastigote form into the host.

T. cruzi species are mainly present in South America, the parasite has a broad host range (including domestic and wild animals), but is famous for causing Chagas disease in man. The parasites are transmitted by cone-nosed bugs (like Rhodnius spp. and Triatoma spp.). The metacyclic trypomastigote stage infects the host and unlike T. brucei, will multiply inside the host cytoplasm of different cell types. After rupture of the host cell new trypomastigote forms are released which can again be ingested by cone nosed bugs.

The phylum Apicomplexa, harbours i.a. parasites of the family Eimeriidae. Many different Eimeria species are present in a large variety of mammals and birds. Seven prevalent species infecting the gastrointestinal tract of chickens are Eimeria tenella, E. necatrix, E. brunetti, E. maxima, E. acervulina, E. praecox and E. mitis. These Eimeria species are all involved in Coccidiosis in poultry. This makes Eimeria the cause of the most important parasitic disease in poultry, causing great economically losses for farmers. Eimeria infect epithelial cells and submucosa of the intestines, causing severe hemorrhagic enteritis, which leads to high mortality in young birds.

This disease has a worldwide distribution and is the most frequently recorded disease affecting poultry kept in modern poultry industry.

The family of Sarcocystidae, comprising Toxoplasma, Sarcocystis and Neospora is also known to have pathogenic members.

Toxoplasma is a widespread parasitic infection, being present in almost all mammals, in particular in goat, sheep and pigs, but also in humans. Prevalence in human populations can be as high as 70% of the total population. Infection often occurs via eating of undercooked meat contaminated with the parasite, but can also occur by ingestion of oocysts, being spread in the faeces of cats, which are the final host. When animals or humans are infected during pregnancy, it can cause spontaneous abortion or congenital toxoplasmosis in the developing foetus. This can result in, neurological sequels or ocular disorders. Chronic and lethal infections (encephalitis) can occur in immune compromised patients.

Neospora, in particular N. caninum is a coccidian parasite very similar to Toxoplasma. However, in contrast to Toxoplasma, Neospora has the dog as final host. N. caninum induces abortions in its intermediate host, and can cause severe abortion storms in cattle. Another Neospora species, N. hughesi, is suspected to cause equine protozoal myeloencephalitis in horses.

Many Sarcocystis species are present in cattle, pigs, sheep, goats and horses. Economically, Sarcocystis neurona is recognized as the most common cause of clinical equine protozoal myeloencephalitis in horses. In the U.S. 50% of horses are seropositive for S. neurona.

Plasmodium belongs to the Haemosporida and is known i.a. as the cause of malaria, being transmitted by mosquitoes. In humans four Plasmodium species have been described, of which P. falciparum is the most pathogenic and deathly. 400 million people are estimated to be infected, causing two million deaths each year. Initial clinical symptoms are rhythmic fevers. After initial infection, Plasmodium parasitsizes the red blood cells, often resulting in anaemia. Parasitsized red blood cells are sequestrated in capillaries of internal organs, thereby causing tissue anoxia. This is particularly serious in the brain, giving rise to multiple petechial haemorrhages, leading to oedema and coma, which may be fatal. Although Plasmodium species have mainly been described in man, other Plasmodium species can infect al large variety of vertebrates.

Babesia and Theileria, both belonging to the Piroplasmida harbour parasite species affecting many mammalian species, and causing a variety of different diseases. Babesia species are transmitted by ticks and can infect a wide range of vertebrates causing a disease referred to as Babesiosis. The disease is characterised by listlessness, anaemia and parasitemia leading to multi-organ dysfunction in infected animals. In advanced stages haemoglobinuria occurs. Important Babesia species in cattle include B. bovis, B. divergens, B. major and B. bigemina. In dogs B. canis, B. rossi, B. microti and B. gibsoni species are mainly causing Babesiosis and are a common cause of death. Some Babesia species, like B. divergens and B. microti have been reported to infect humans as well.

Theileria is a tick-transmitted disease, infecting ruminants and is mainly a problem in cattle. Theileria infects and develops in leukocytes and erythrocytes. Pathology is mainly attributable to the intraleukocyte stage. Two major Theileria species should be discriminated in cattle, T. parva and T. annulata. T. parva causes East Coast Fever, a deathly cattle disease, being endemic in various African countries. East Coast Fever is characterised by high fever, lymphadenopathy, severe pulmonary oedema and wasting. T. annulata infects cattle and buffalo, first invading cells of the lymphatic system and later appearing in the peripheral blood as intra-erythrocyte forms. Infection with T. annulata is usually referred to as tropical Theileriosis. The disease starts with high fever and swelling of lymph nodes, followed by listlessness, accelerated pulse and respiration rates and anorexia. In the final stage of disease anaemia is observed and ultimately death occurs. In the horse Babesia equi (which has been re-named as Theileria equi) is also a major pathogen.

It is clear that different ways of attack against these parasites have been studied through the years.

One of the routes of combating parasitic infections is the use of pharmaceutical components, such as the extensive use of anticoccidials that nowadays is a very common therapy in the treatment of poultry Coccidiosis. Another route is undoubtedly vaccination. It is clear that, especially where there is an increasing reluctance against the use of antibiotics, there is a need for new and effective vaccines, especially vaccines that provide broad protection.

Currently, two different approaches are used in vaccination against parasitic infections: vaccination with a live attenuated vaccine and vaccination with inactivated (killed) vaccines. Both approaches have their advantages and disadvantages, as summarized below:

The main advantage of attenuated vaccines is that they closely mimic the natural infection: they activate all phases of the immune system, they can induce humoral IgG and local IgA, they raise immune responses to many protective antigens, they provide a more durable immunity and are more cross-reactive. Moreover they are low-cost and they provide a quick immunity in the majority of cases.

Disadvantages of attenuated vaccines are the difficulties in finding the right level of attenuation and the possibility of reversion to virulence (these are major disadvantages), the spread to contacts of the vaccinee and the problems in immuno-compromised humans and animals.

Advantages of inactivated vaccines are that they provide sufficient humoral immunity if boosters are given, they show no mutation or reversion (a big advantage), they can be used with immuno-deficient patients, and in principle they are safe.

Disadvantages of inactivated vaccines: they often do not raise (cellular) immunity, boosters are needed, they provide no local immunity (important), they are more expensive and their use is dangerous if Inactivation is below 100%.

Development of vaccines against parasites however is complex, if only because of the complexity of the parasites as such, when compared to other microorganisms. Next to this, the various parasites even within the phylum Apicomplexa and within the family of Trypanosomatidae, although related, do not have sufficient similarity in their genetic make-up to allocate a common attenuation site or inactivation method, equally applicable to all these parasites. Moreover, for the manufacture of attenuated live vaccines it is necessary to locate suitable attenuation targets for each and every parasite. For the production of killed vaccines, one needs to know which antigens must be left unaltered by the inactivation method for each and every parasite. And apart from this, so far, many inactivated parasite vaccines have been shown not to be effective. Finally, there is a variety of different infection routes, different hosts, different host cells within the host and often even host changes during the life cycle which is a characteristic of most parasites and which again differs from parasite to parasite. This also complicates the development of vaccines.

Therefore, the development of vaccines for combating parasitic infection so far has been difficult, time consuming and not very successful.

It is an objective of the present invention to provide vaccines for combating infections caused by parasites of the phylum Apicomplexa and the family of Trypanosomatidae, that combine most of the advantages of both killed and live attenuated vaccines almost completely without having the disadvantages of these vaccines. Moreover, the method for the production of such vaccines is universally applicable to parasites of the phylum Apicomplexa and the family of Trypanosomatidae.

In the life cycle of all parasites of the phylum Apicomplexa and the family of Trypanosomatidae, there is at least one moment in which a certain stage infects a cell of a host and starts dividing. It was now surprisingly found that if ribosome synthesis can be stopped at or around the moment of infection, the parasite nevertheless does enter the host cell and divides several times using the present pool of ribosomes, thereby perfectly mimicking natural infection. Finally however, after several rounds of dividing, the progeny parasites will die due to lack of ribosomes.

This has the advantage that the induction of the immune response after infection is triggered in the most natural way, as if a virulent infection occurred, whereas contrary to the natural situation the parasite will after some time unavoidably become extinct. This goal was attained by placing a ribosomal protein gene under the control of an inducible promoter.

An inducible promoter is a promoter that can deliberately be switched on and off. Examples of such promoters will be given below.

In principle, each ribosomal protein gene can be used as a target, since in principle all ribosomal proteins are needed for the synthesis of a stable, fully functional ribosome. All parasites of the phylum Apicomplexa and the family of Trypanosomatidae have cytoplasmatic ribosomes, and most of them have plastid ribosomes and/or mitochondrial ribosomes. All of these are necessary for the normal development of the parasite. Therefore, live attenuated parasites according to the invention can be obtained by placing a ribosomal protein gene under the control of an inducible promoter, regardless the fact if this ribosomal protein gene encodes a ribosomal protein to be incorporated in plastd-, mitochondrial or cytoplasmatic ribosomes.

Ribosomal protein sequences are highly conserved between the various parasites. Therefore, DNA probes of the ribosomal sequences provided below can be used for the detection of the analogous ribosomal proteins in each of the parasites of the phylum Apicomplexa and the family of Trypanosomatidae. Additionally, the sequences of many ribosomal protein genes for many different parasites can be found in the NCBI-protein database (http://www.ncbi.nlm.nih.gov).

The fact that the lack of one ribosomal protein can already disturb the formation of stable ribosomes has been demonstrated in various plants, animals and microorganisms. Merely as an example: in Drosophila, mutations in some of the eighty ribosomal proteins have been shown to result in a typical phenotype, e.g. thin and short bristles, prolonged development, and female semi-sterility in heterozygotes as well as homozygous lethality. This phenotype, termed Minute phenotype, has been observed with mutations in for example the ribosomal proteins S13, and L9, (Schmidt, A., Hollmann, M., Schäfer, U., Mol. Gen Genet. 251:381-387 (1996), Sæbøe-Larssen & S., Lambertsson, A., Genetics 143: 877-885 (1996)). Another example is the ribosomal protein gene YS3 of yeast, which encodes the yeast ribosomal protein S3. Its disruption yields non-viable haploid spores of Saccharomyces cerevisiae (Finken-Eigen, M., Domdey, H., Köhrer, K., Biochemical and Biophysical research communications 223, 397-403 (1996)). These studies demonstrated that down-regulating a single ribosomal protein can already affect the formation and/or proper functioning of ribosomal complexes.

The promoters to be used in parasites according to the invention for the control of transcription of the ribosomal protein gene need to fulfil only one prerequisite. They must be switched on during the propagation of the parasites. This is of course necessary in order to provide the parasite according to the invention with the native amount of ribosomes necessary for normal propagation. The promoter must however be switched off in the recipient host that receives the parasite as a vaccine. A promoter is considered to be switched on if it initiates the transcription of the gene it controls. In the present invention this gene would be a ribosomal protein gene. A promoter is switched off if transcription of the gene that it controls is at least two times lower than in the on situation. Preferably, the level of transcription is at least 3, more preferably 4, still more preferably 5, 6 or even 7 times lower. It is stressed, that there is no need for a complete inhibition of transcription anyway. A low level of ribosomal protein transcription will finally result in an extended live span of the parasites, before they become extinct. Thus they will trigger the immune system for a somewhat longer period.

In principle, there are two different possibilities: either the promoter is switched on unless some condition is applied that switches the promoter off, or the promoter is switched off unless some condition is applied that switches the promoter on.

Preferably, the promoter is in the switched off status unless some condition is applied that is not present in the recipient host that receives the parasite as a vaccine.

If necessary, two or more ribosomal protein genes can be placed under the control of inducible promoters. This would be a preferred option if the inducible promoter used in a promoter cannot be sufficiently switched off, i.e. if the inducible promoter is a leaky promoter, or in the exceptional case that lack of one specific ribosomal protein is not sufficient to destabilize the ribosome.

The invention will be explained by the following examples.

Toxoplasma gondii uses the cat as a final host, and uses herbi- and omnivores respectively carnivores as subsequent intermediate hosts. In the case of Toxoplasma, it is the oocyst/tissue cyst stage of the parasite that ultimately infects humans. Humans and warm-blooded animals are the target mammals for vaccination, and therefore the Toxoplasma tachyzoite is the parasitic stage for which the live attenuated parasite is needed. Therefore, the tachyzoite is the parasitic stage in which, according to the invention, a ribosomal protein gene is brought under the control of an inducible promoter. The thus made recombinant parasite, further also referred to as the attenuated live parasite, can be propagated in the classical way under conditions under which the promoter is switched on. Under these circumstances, the number of ribosomes will be identical or close to that in the native situation. If sufficient parasites are grown for vaccine purposes, the live attenuated parasites are collected and administered as a vaccine. In the host to be vaccinated, the conditions under which the promoter is switched on are not present and as a result the promoter will remain in the switched off situation. At the moment of vaccination, the parasite will behave as a wild-type parasite, because the pool of ribosomes is comparable to the native situation. Therefore, the process of infection, and of invasion of the host cell will perfectly mimic the process of natural infection. As soon as the parasite starts dividing in the host, it also divides the pool of ribosomes over its progeny. Since the promoter of (at least) one of the ribosomal protein genes is however in the switched off position when in the host cell, there will be either reduced or even no de novo synthesis of ribosomes. Therefore, the progeny will slowly become extinct. Nevertheless, in the meantime the process of infection, and therefore the triggering of the immune system has continued as in the case of a wild-type parasitic infection. Therefore, ultimately immunity will have build up as if an infection with a virulent wild-type parasite had taken place, whereas the live attenuated parasites used for the induction of immunity have become extinct after one or a few rounds of infection. The examples below provide further details.

The life cycle of Neospora caninum is comparable with that of Toxoplasma except for the fact that Neospora uses dogs as the final host, and causes abortions in i.a. cattle, dogs, sheep and horses. The approach for Neospora vaccines thus closely relates to that of Toxoplasma as described above. As for Toxoplasma, the tachyzoite is the parasitic stage in which, according to the invention, a ribosomal protein gene is brought under the control of an inducible promoter. The development of molecular genetics tools for Neospora has been described i.a. by Howe, D. K. and Sibley, L. D. METHODS: 13(2): 123-33 (1997))

For the production of a life attenuated Eimeria parasite, the merozoite is the parasitic stage in which, according to the invention, a ribosomal protein gene is brought under the control of an inducible promoter. In this case, the vaccine does not comprise the merozoite however, but the sporulated oocysts. This is due to the fact that the sporulated oocyst is the form in which the parasite is normally ingested by the chicken. For the replication of the first recombinant merozoites made according to the invention, it suffices however to introduce these into the digestive tract of the chicken. Recombinant oocysts will then be shed by the chicken and can be isolated and directly used as the live attenuated parasite in Coccidiosis vaccines, e.g. oral vaccines for administration to drinking water. Isolation of oocysts from chicken dung is a standard procedure well known in the art. Genetic engineering of Eimeria has i.a. been described by Kelleher, M. and Tomley, F. M. (Mol. Biochem. Parasitol. 97(1-2): 21-31 (1998)).

A live attenuated Malaria vaccine according to the invention can be made e.g. starting from erythrocyte stage plasmodium parasites. Plasmodium recombinant sporozoites. The sporozoite is the phase of the parasite that is injected into the (human) blood stream by the female mosquito. The sporozoite infects the liver within two minutes after injection, to produce schizonts and merozoites. The merozoites, in turn, infect erythrocytes and replicate there. It is at this moment in time that the pool of ribosomes must be divided over a large number of progeny parasites, and this is the moment at which the progeny parasites will become extinct. The whole immune defence system is already fully triggered at that moment in time. This example again illustrates the advantage of vaccines based upon recombinant parasites according to the present invention: they share all the advantages of live vaccines with the advantages of inactivated vaccines. Vaccination will preferably be done with either recombinant erythrocyte stage plasmodium parasites or (less practically) recombinant sporozoites. Recombinant DNA techniques for Plasmodium have been described i.a. by Crabb, B. S., et al., (Mol. Biochem. Parasitol. 90: 131-144 (1997)) and by Wu, Y. et al., (Proc. Natl. Acad. Sci., 93: 1130-1134 (1996), and Proc. Natl. Acad. Sci., 92: 973-977 (1995))

Live attenuated Theileria vaccines according to the invention can again be based upon recombinant merozoites. These merozoites can be grown and maintained in lymphocytes. It is in the lymphocyte that the merozoite starts dividing, synchronously with the division of the lymphocyte, while a few free progeny parasites will infect other lymphocytes, altogether leading to the induction of wild type like immunity, however leading, as in the other examples, to progeny that finally becomes extinct due to slowly increasing lack of ribosomes. Theileria can be propagated and cultured in primary lymphoid cells. See e.g. Shkap V. et al., Vet. Parasitol. 65: 11-20 (1996) and Hulliger, L. J. Protozool. 12: 649-655 (1965).

Live attenuated Babesia vaccines can be made using the merozoites and/or trophozoites for recombination. These can be cultured in erythrocytes. The whole approach is comparable to that described for Theileria above. See i.a. Levy, M. G and Ristic, M. Science 207: 1218-1220 (1980).

For Sarcocystis species such as S. suihominis and S. neurona, both the sporozoite and the merozoite are targets for recombination according to the invention. And again, the principle is the same: the recombinant sporozoite provides recombinant merozoites and these merozoites slowly become extinct due to lack of ribosomes in the absence of de novo ribosome protein synthesis. The recombinant merozoites can be used directly in a vaccine. See e.g. Murphy, A. J. and Mansfield, L. S. J. Parasitol. 85: 979-981 (1999) and Ellison, S. P. et al., Vet. Parasitol. 95: 251-261 (2001).

As far as the order of Kinetoplastida is concerned, tetracycline regulated gene expression has been described for Trypanosoma brucei (Wirtz, E. and Clayton, C., Science 268: 1179-1183 (1995) and Biebinger, S. et al., Mol. & Biochem. Parasitol. 85: 99-112 (1997)); Trypanosoma congolense (Inoue N., et al., Mol. & Biochem. Parasitol. 120: 309-313 (2002)) and Leishmania donovani (Yan, S., et al., Mol. & Biochem. Parasitol. 112: 61-69 (2001)), and can be adjusted to regulate ribosomal protein gene transcription as follows: briefly, the procyclic form of the parasite is the target for transfectons. The tetracycline repressor is integrated into the non-transcribed spacers of the ribosomal RNA repeats, so that heterologous genes (in this reference not a ribosomal gene) can be regulated in a tetracycline dependent manner. For the construction of live attenuated parasites according to the invention of the order of Kinetoplastida, first an extra copy of a ribosomal protein gene is inserted together with a promoter containing one or more tetracycline operator elements. Subsequently, the endogenous gene copy is deleted from the parasite genome. This can easily be done by homologous recombination preferably in the presence of a marker for recombination. This is comparable to methods for Apicomplexa as described below. Direct targeting of the endogenous ribosomal protein genes is not feasible for Leishmania and Trypanosoma, because most genes in Leishmania and Trypanosomes are organized into large (>100-500 kb) polycistronic clusters of adjacent genes on the same DNA strand. Thus inhibition of one gene would lead to inhibition of the transcription of all the genes localised downstream (Myler, P. J. et al., Med. Microbiol. Immunol. 190: 9-12 (2001)).

The examples given above are indeed merely examples. They by no means limit the scope of the invention. Examples of all kinds of parasites of the phylum Apicomplexa and the family of Trypanosomatidae and their life cycles can be found in the Encyclopaedic Reference of Parasitology, Heinz Mehlhorn, Springer Verlag (2001) (ISBN 3-540-66829-2). Man skilled in the art is thus perfectly able, with the examples given above and using the Encyclopaedic Reference of Parasitology, to determine which stage would be the preferred stage as a starting point for making the live attenuated parasite according to the invention, for each parasite of the phylum Apicomplexa and the family of Trypanosomatidae.

Many of the parasites belonging to the families mentioned above have a variety of different hosts. Merely as an example: there are Babesia species such as B. canes infecting dogs, B. caball infecting horses, mules and donkeys, B. divergens infecting cattle, wild ruminants and humans. Nevertheless, in all cases the parasitic life cycle is comparable. Therefore, where it is Indicated above that a vaccine according to the invention against e.g. Babesia can be based upon recombinant merozoites, this holds true for all Babesia species. Details concerning the life cycles of the various species of one family can also be found in the Encyclopaedic Reference of Parasitology, Heinz Mehlhorn, Springer Verlag (2001) (ISBN 3-540-66829-2), mentioned above.

Thus, one embodiment of the present invention relates to attenuated live parasites of the phylum Apicomplexa or the family of Trypanosomatidae that have as a characteristic that they comprise a ribosomal protein gene under the control of an inducible promoter.

The concept of inducible promoters has already been mentioned shortly above. An inducible promoter is a promoter that can be switched on and off under the influence of an external factor. Such a switching factor can be a physiological factor e.g. heat; the trigger of all of the many heat-shock promoters well known in the art for decades already. Such a factor can also be of chemical nature. Many such factors are again well known in the art. There are too many inducible promoters known in the art to mention them all. A few examples will be mentioned here. The IPTG-inducible Lac-promoter is possibly one of the most frequently used inducible promoters. Alternative inducible promoter systems are e.g. the tetracycline-controlled transactivation system (Baron, U. et al., Oxford University Press 25: 2723-2729 (1995)) and the ecdysone-inducible expression system (Invitrogen) (Yao, T. P. et al., Cell 71: 63-72 (1992)).

In principle there are two kinds of inducible promoters: those that are switched on in the presence of a condition, and those that are switched off in the presence of a condition. This condition may be the presence of a chemical substance.

In a preferred form of this embodiment of the invention, the promoter to be used is switched on in the presence of a condition that is not naturally present in the host. The use of such promoters has the advantage that they automatically are in (or will switch to) the switched off position as soon as they are administered to the natural host of the parasite. This implies that a live attenuated parasite according to the invention is preferably grown under “artificial” conditions, i.e. conditions not present in the natural host, in order to replicate.

A preferred type of inducible promoters is the type of inducible promoters based upon an operator site and a repressor capable of reversibly binding said operator site. The binding and detachment of the repressor protein can then be regulated by the “condition” applied as mentioned above, i.e. the presence or absence of heat, chemicals, etcetera.

A very suitable example of an inducible promoter, or more precisely; a promoter/operator/repressor complex, that can very efficiently be used in attenuated live parasites according to the invention, is the tet-promoter/tet-operator complex, further also referred to as the tetR-system.

The tetR-system as such has been described and proven to work in many different protozoan parasites, such as T. brucei (Wirtz et al., Science 268:1179-1183 (1995), Biebinger et al. (Mol. Biochem. Paras.85: 99-112 (1997)) and in Entamoeba hystolitica (Hamann et al., Mol. Biochem. Paras. 84: 83-91 (1997)). The tetR-system was also successfully used in Toxoplasma to regulate expression of myosin A (Meissner M, et al., Nucleic Acids Res. 29(22): E115 (2001)). In addition, tetracycline regulated expression was also demonstrated in Giardia lambila and Leishmania donovani, showing its universal applicability in parasites (Yan S, etal., Mol Biochem Parasitol. 112(1): 61-9 (2001), Sun, C. H. and Tai, Mol. Biochem. Parasitol. 105(1): 51-60 (2000)).

This complex operates as will be described shortly below and more extensively in the examples.

In principle, two steps must be made in order to generate tetracycline-regulated expression of ribosomal proteins: 1. integration and expression of a tetracycline repressor (tetR) gene and 2. integration of one or more tetracycline operator element(s) in the promoter of a ribosomal protein gene near the start of transcription. The tet-repressor gene is a gene that encodes a protein capable of binding to the tet-operator site thus blocking transcription of the adjacent gene. This gene is now placed under the control of a constitutive promoter (i.e. constitutive in the recombinant parasite) and brought into the parasite using recombinant DNA techniques. Thus, the recombinant parasite will synthesize the tet-repressor protein. The tet-operator is preferably introduced in the vicinity of the transcription start site of one or more ribosomal protein genes, preferably in the endogenous promoter, upstream of the STS. The tet-repressor protein will consequently bind to the tet-operator, thus blocking the transcription of the downstream ribosomal protein gene. In the presence however of tetracycline, the repressor will detach from the tet-operator site, thus enabling the transcription of the downstream gene. Therefore, in the presence of tetracycline, the recombinant parasite will be able to replicate as in the natural situation. If the recombinant parasite can be grown in vitro, as is the case for many parasites including most of the parasites of the examples given above, tetracycline can easily be added to the growth medium. If the growth of the parasites requires propagation in the natural host, which is e.g. the case for Eimeria parasites, tetracycline can easily be administered orally or by injection to the host (in this case the chicken). The following should be noted: tetracycline is taken up by extracellular and intracellular parasites. Cell rupture of the host cell is not required for the drug to have effects on the regulation of the expression of ribosomal proteins.

Step 1, the integration and expression of the tetracycline repressor gene (tetR), can be obtained as described in the literature mentioned above. A suitable and well-known selection marker that indicates the stable transformation and possibly integration of the tetracycline repressor gene is e.g. the CAT-gene (Kim, K., et al., Science 262(5135): 911-4 (1993)). Other suitable markers for selection of stable transfection are also known in the art, such as DHFR-TS (Donald, R. G. and Roos, D. S., Proc. Natl. Acad. Sci. U S A 90 (24): 11703-11707 (1993), Roos, D. S. et al., METHODS 13:112-122 (1997)) and HXGPRT (Donald, R. G. et al., J. Biol. Chem. 271: 14010-14019 (1996), Donald, R. G. and Roos, D. S., Mol. Biochem. Parasitol. 91 (2): 295-305 (1998)).

The Cre-lox system also provides a suitable selection system (see i.a. Hardy, S. et al., Joum. Virol. 71: 1842-1849 (1997)).

If the tetR-system is used as an inducible promoter system, the promoter upstream of the ribosomal protein gene can e.g. be the endogenous promoter, now made inducible by cloning the tet-operator in the vicinity of the start site of transcription (see below for details of the tet-operator sequence and preferred insertion sites). It goes without saying that any other promoter capable of providing a sufficiently high transcription level of the ribosomal protein gene is also suitable.

If another Inducible promoter system is used, it would be easy to use that inducible promoter and delete the endogenous promoter. If however another regulatory element is used, of which the principle is comparable to the tet-operator, the promoter itself can equally well be the endogenous promoter. Again it goes without saying that any other promoter capable of providing a sufficiently high transcription level of the ribosomal protein gene cloned downstream, Is also suitable.

Step 2, the replacement of a wild-type ribosomal protein gene with one containing one or more tetO sites (=tet-operator sites) in the vicinity of the STS requires the insertion of the tet-operator site between the promoter of the ribosomal protein gene of choice and the gene itself. The tet-operator has been described by Yan S, et al. (Mol. Biochem. Parasitol. 112(1): 61-9 (2001)), by Wirtz, E and Clayton, C. (Science 268(5214): 1179-83 (1995and by Meissner M, et al. (Nucleic Acids Res. 29(22): E115 (2001)).

The sequence of a single tet operator (tetO) site is

5′-TCCCTATCAGTGATAGAGATC-3′.

In principle, insertion of a single tet-operator site in front of the ribosomal protein gene of choice would suffice. The tetR-system is, as all biological systems, however not inducible from exactly 0% to 100% activity and vice versa. Therefore, if a stronger level of regulation is needed, preferably two or more operator sites are inserted. The tet-operator interferes with the binding of the RNA-polymerase that transcribes the downstream gene. Therefore, the tet-operator is preferably inserted somewhere in the region that extends from nucleotide −100 to +3 relative to the site at which the transcription starts (herein referred to as the STS). Moreover, in the examples it is additionally described how to locate such STS.

The step of replacement of a wild-type ribosomal protein gene with a recombinant gene comprising one or more tet-operator sites can i.a. be performed with the hit-and-run strategy described by Donald, R. G. and Roos, D. S. (Mol. Biochem. Parasitol. 91(2): 295-305 (1998)).

The skilled artisan will be able to find alternative methods using other combinations of positive and negative selection markers. HSV Thymidine kinase can for example be used as a negative selection marker. (LeBowitz, J. H. et al., Mol. Biochem. Parasitol. 51(2): 321-5 (1992), Fox, B. A. et al, Mol. Biochem. Parasitol. 116(1): 85-8 (2001)).

The molecular tools used for the construction of recombinant Toxoplasma parasites according to the invention work equally well in Neospora (Howe, D. K. and Sibley, L. D. METHODS 13(2): 123-133 (1997)).

In Eimeria, the same methods are equally applicable. Merely as an example: it was shown that beta-galactosidase could be transiently expressed in E. tenella by Kelleher, M. and Tomley, F. M. (Mol Biochem Parasitol. 97(1-2): 21-31 (1998)). For Theileria, methods have e.g. been developed to transiently transfect infective, uninucleate Theilera annulata sporozoites by Adamson, R. et al. (Mol. Biochem. Parasitol. 114(1): 53-61 (2001)).

In Plasmodium, dihydrofolate reductase-thymidylate synthase (dhfr-ts) coding sequences mutated to confer resistance to pyrimethamine, or Puromycin-N-acetyltransferase, or the blasticidin S deaminase (BSD) gene of Aspergillus, or the neomycin phosphotransferase II (NEO) gene from transposon Tn5 have been described as selectable markers (Wu, Y., et al., Proc. Natl. Acad. Sci. U S A. 93(3): 1130-4 (1996), Wang, P., et al., Mol. Biochem. Parasitol. 123(1): 1-10 (2002), de Koning-Ward, T. F., et al. (Mol. Biochem. Parasitol. 117 (2):155-60. (2001))

Similar selection markers work in Babesia as well.

Therefore, man skilled in the art will be able to apply the present invention over the full range of parasites belonging to the phylum Apicomplexa and the family of Trypanosomatidae.

A preferred form of this embodiment relates to an attenuated live parasite according to the invention that belongs to the Coccidia, the Piroplasmida or the Haemosporida.

In a more preferred form of this embodiment, the attenuated live parasite belongs to the family Eimeridiidae, Cryptosporidiidae or Sarcocystidae.

In an even more preferred form of this embodiment, the attenuated live parasite belongs to the genus Eimeria, Cryptosporidium, Toxoplasma, Sarcocystis or Neospora.

In another more preferred form of this embodiment, the attenuated live parasite belongs to the family of the Babesiidae or the Theileriidae.

In an even more preferred form of this embodiment, the attenuated live parasite belongs to the genus Babesia or Theileria.

In another more preferred form of this embodiment, the attenuated live parasite belongs to the genus Plasmodium.

In still another more preferred form of this embodiment, the attenuated live parasite belongs to the genus Trypanosoma or the genus Leishmania.

In an even more preferred form, the attenuated parasite belongs to the species Leishmania mexicana, L. infantum or L. major or the species Trypanosoma brucei or T. cruzi

In another preferred form of this embodiment, a ribosomal protein gene of the live attenuated parasite according to the invention is under the control of an inducible promoter that is inducible by antibiotics.

More preferably, these antibiotics are tetracycline or anhydrotetracyclin, or derivatives thereof.

In another preferred form of this embodiment, the ribosomal protein gene of choice is the gene encoding L9, S3, plastid-S9 or S13, preferably the L9, S3, plastid-S9 or S13 of Toxoplasma gondii.

The nucleotide sequence of the gene encoding Large subunit ribosomal protein number 9 (L9), as well as upstream sequences comprising the promoter region is depicted in SEQ ID NO: 1

| | | | | |
|---|---|---|---|---|
| REGION | 1 | 2296 | promoter | promoter region |
| REGION | 2297 | 2461 | e | exon 1 |
| REGION | 2416 | 2418 | atg | atg start codon |
| GENE | 2416 | 4831 | cds | coding sequence |
| REGION | 2462 | 3838 | i | intron 1 |
| REGION | 3839 | 4260 | e | exon 2 |
| REGION | 4261 | 4727 | i | intron 2 |
| REGION | 4728 | 4834 | e | exon 3 |
| REGION | 4829 | 4831 | stop | TAA stopcodon |

The nucleotide sequence of the gene encoding plastid Small subunit ribosomal protein number 9 (S9), as well as upstream sequences comprising the promoter region is depicted in SEQ ID NO: 2

| | | | | |
|---|---|---|---|---|
| REGION | 1 | 3076 | promoter | promoter region |
| REGION | 3077 | 3616 | e | exon 1 |
| REGION | 3156 | 3158 | atg | ATG start codon |
| GENE | 3156 | 4325 | cds | coding sequence |
| REGION | 3617 | 3874 | i | intron 1 |
| REGION | 3875 | 4034 | e | exon 2 |
| REGION | 4035 | 4130 | i | intron 2 |
| REGION | 4131 | 4338 | e | exon 3 |
| REGION | 4323 | 4325 | stop | TAG stop codon |
| REGION | 4326 | 4338 | 3′ utr | 3′ UTR |

The nucleotide sequence of the gene encoding Small subunit ribosomal protein number 13 (S13), as well as upstream sequences comprising the promoter region is depicted in SEQ ID NO: 3

| | | | | |
|---|---|---|---|---|
| REGION | 1 | 1289 | promoter | promoter region |
| REGION | 1290 | 1594 | e | exon 1 |
| REGION | 1448 | 1450 | atg | ATG start codon |
| GENE | 1448 | 3639 | cds | coding sequence |
| REGION | 1595 | 2527 | i | intron 1 |
| REGION | 2528 | 2615 | e | exon 2 |
| REGION | 2616 | 3489 | i | intron 2 |
| REGION | 3490 | 3639 | e | exon 3 |

The nucleotide sequence of the gene encoding Small subunit ribosomal protein number 3 (S3), as well as upstream sequences comprising the promoter region is depicted in SEQ ID NO: 4

| | | | | |
|---|---|---|---|---|
| REGION | 1 | 1177 | promoter | promoter region |
| REGION | 1178 | 1308 | e | exon 1 |
| REGION | 1291 | 1293 | atg | ATG start codon |
| GENE | 1291 | 2651 | cds | coding sequence |
| REGION | 1309 | 1752 | i | intron 1 |
| REGION | 1753 | 2137 | e | exon 2 |
| REGION | 2138 | 2249 | i | intron 2 |
| REGION | 2250 | 2389 | e | exon 3 |
| REGION | 2390 | 2486 | i | intron 3 |
| GENE | 2487 | 2748 | e | exon 4 |
| REGION | 2649 | 2651 | stop | TAA stop codon |
| REGION | 2652 | 2748 | 3′ utr | 3′ UTR |

Attenuated live parasites according to the invention are very suitable for use in vaccines. This is, as extensively explained above, due to the fact that they combine the advantages of both live attenuated and inactivated vaccines, without suffering from the disadvantages. Therefore, another embodiment of the present invention relates to attenuated live parasites according to the invention for use in a vaccine.

Still another embodiment of the invention relates to vaccines for combating parasitic infection that comprise a live attenuated parasite according to the invention and a pharmaceutically acceptable carrier.

A pharmaceutically acceptable carrier can be e.g. sterile water or a sterile physiological salt solution. In a more complex form the carrier can e.g. be a buffer such as PBS, well-known in the art.

Vaccines according to the present invention may in a preferred presentation also contain an immunostimulatory substance, a so-called adjuvant. Adjuvants in general comprise substances that boost the immune response of the host in a non-specific manner. A number of different adjuvants are known in the art. Examples of adjuvants frequently used in cow vaccines are muramyldipeptides, lipopolysaccharides, several glucans and glycans and Carbopol® (a homopolymer).

The vaccine may also comprise a so-called “vehicle”. A vehicle is a compound to which the protein adheres, without being covalently bound to it. Such vehicles are i.a. lipid vesicles, ISCOMs®, dendromers, niosomes, microparticles, especially chitosan-based microparticles, polysaccharide matrices and the like, bio-microcapsules, micro-alginates, liposomes and macrosols, all known in the art. Microparticles, more specifically those based upon chitosan, especially for use in oral vaccination are very suitable as vaccine vehicles.

A special form of such a vehicle, in which the antigen is partially embedded in the vehicle, is the so-called ISCOM® (EP 109.942, EP 180.564, EP 242.380)

In addition, the vaccine may comprise one or more suitable surface-active compounds or emulsifiers, e.g. Span® or Tween®. Also, the vaccine may comprise one or more immune stimulants such as cytokines, e.g. interferons.

Vaccines based upon live attenuated recombinant parasites described above can be administered in relatively low amounts, when compared to inactivated parasites, because they multiply themselves during the infection. Therefore, very suitable amounts would range between 10² and 10⁷ parasites per dose. Amounts below 10² parasites per dose may not always guarantee a sufficient level of protection in all vaccinated animals. Ranges from 10⁷ up to 10⁸ parasites per dose, although suitable, are not very practical, if only from an economic point of view.

Still another embodiment of the present invention relates to the use of an attenuated live parasite according to the invention for the manufacture of a vaccine for combating infection caused by a parasite of the phylum Apicomplexa or the family of Trypanosomatidae.

Again another embodiment of the present invention relates to methods for the production of a vaccine according to the invention that comprise the mixing of a live attenuated parasite according to the invention and a pharmaceutically acceptable carrier.

Vaccines according to the invention can be administered e.g. intradermally, subcutaneously, intramuscularly, intraperitoneally, intravenously, or at mucosal surfaces such as orally or intranasally.

The tet-repressor gene is a gene of prokaryotic origin. The codon usage for this gene is consequently sub-optimal in eukaryotic organisms such as the live attenuated parasites to which the present invention relates. Therefore, man skilled in the art would be motivated to adapt the coding sequence of the tet-repressor gene in such a way that it corresponds to the codon usage of the eukaryotic cell, thus arriving at a synthetic tet-repressor gene. This has been done by Meissner M, et al. (Nucleic Acids Res. 29(22): E115 (2001)). Of course one would expect that this synthetic tet-repressor gene could not be further optimised, since it is already fully adapted to the eukaryotic cell. Moreover, one would expect this “synthetic” tet-repressor protein to be the best suitable repressor protein in the eukaryotic cell. This protein is in principle the same protein as the native protein, and thus by definition best fitted for interaction with the tet-operator site.

It was however surprisingly found now, that fusion proteins encoded by a recombinant gene comprising (part of) a heterologous gene fused to the N-terminal part of the native i.e. prokaryotic tet-repressor provide a significantly better regulation of the tet-operator than even the tet-repressor protein encoded by a fully eukaryote-adapted “synthetic” tet-repressor gene.

Thus, such fusion proteins would be the repressor proteins of choice to be used in the live attenuated parasites according to the present invention. This is even more an unexpected finding because 3D-structure studies of the tet-repressor protein would predict that N-terminal fusion would negatively interfere with DNA-binding. This was however surprisingly found not to be the case in practice.

A heterologous gene is any gene that encodes a protein other than the tet-repressor protein. A heterologous protein is any protein other than the tet-repressor protein. A recombinant gene is any artificially made gene that comprises (part of) a heterologous gene fused to that side of the tet-repressor gene that encodes the N-terminus of the tet-repressor protein.

The fusion protein must be able to reach the nucleus in order to interact with the tet-operator. Therefore there are a number of prerequisites to be fulfilled by the tet-repressor fusion protein: the final molecular weight of the monomeric tet-repressor fusion protein must be <60 kD, the heterologous part of the fusion protein must be on the N-terminal side of the tet-repressor protein, and the fusion protein must be free of GPI-anchors, secretion/excretion signals and trans-membrane regions. In principle, each and every protein or part thereof that meets with these prerequisites and (as a consequence) is capable of targeting the nucleus can be used for N-terminal fusion with the tet-repressor protein.

There is no need to use a full length heterologous protein for fusion. It suffices to use a part of such a heterologous protein. A part is considered to be a fragment of at least 10 amino acids, preferably a least 20 amino acids as the heterologous fusion protein. Preferably, the part originates from the N-terminal side of the heterologous protein. Heterologous proteins of choice are e.g. Green, Red and Yellow Fluorescent protein and the CAT-protein.

Therefore, another embodiment of the present invention relates to DNA-fragments encoding a tet-repressor fusion protein that has as a characteristic feature that it comprises the tet-repressor protein and a heterologous protein or a part thereof, that is fused at the N-terminal side of the tet-repressor protein wherein the monomeric form of the fusion protein has a size of <60 kD and the fusion protein is free of GPI-anchors, secretion/excretion signals and trans-membrane regions.

Still another embodiment of the present invention relates to a tet-repressor fusion protein as such, that has as a characteristic feature that it comprises the tet-repressor protein and a heterologous protein or a part thereof, that is fused at the N-terminal side of the tet-repressor protein wherein the monomeric form of the fusion protein has a size of <60 kD and the fusion protein is free of GPI-anchors, secretion/excretion signals and trans-membrane regions.

The membranes to which the wording “trans-membrane regions” refers, are those membranes that are located between the cytoplasm of the cell and the outside world. These membranes specifically exclude the membranes between the nucleus and the cytoplasm. Preferably, the tet-repressor fusion protein according to the invention does have some signals that specifically direct the fusion protein to the nucleus. This is clear, because the tet-repressor fusion protein (as is required for the native tet-repressor gene) has to enter the nudeus in order to be able to regulate the transcription of the gene it controls.

Due to its universal character, the combination of the tetR-system and the tet-repressor fusion protein can be used not only in live attenuated parasites according to the invention, but certainly also in other parasites and in other eukaryotic cells and organisms. It is universally applicable in eukaryotic cells, for the regulation of expression of any gene.

Attenuated live parasites according to the invention are thus even more suitable as a basis for vaccines, when such parasites comprise the tet-operator combined with (the genetic information encoding) the tet-repressor fusion protein described above. This allows an even better blocking and induction of the transcription of a ribosomal gene.

Therefore, in a more preferred form, attenuated live parasites according to the invention in which the induction of the gene is regulated by tetracycline, anhydrotetracyclin or derivatives thereof, comprise the tet-operator and the genetic information encoding a tet-repressor fusion protein as described above.

As will be shown in the examples, the unexpected characteristics of the tet-repressor fusion protein as described above are even more significant if two or more tet-operator sites are cloned in tandem. The wording “in tandem” should be interpreted broadly, in the sense that tet-operator sites may be cloned directly adjacent to each other or with a spacer sequence in between the two or more tet-operator sites. As mentioned before, the tet-operator sites are preferably cloned in the region between −100 and +3 relative to the STS.

Thus, in an even more preferred forrn, such attenuated live parasites according to the invention comprise not only the tetR-system and a tet-repressor fusion protein as described above, but also two or more tet-operator sites, instead of one.

### EXAMPLES

### Example 1

Primers Used During the Course of the Experiments:

Restriction sites that were inserted are underlined.

| | | | |
|---|---|---|---|
| SEQ ID | | | |
| NO: | # | NAME | SEQUENCE 5′ → 3′ |

| | | | |
|---|---|---|---|
| 5 | 1 | SAG3-FW | CGATAAGCTTCGAATCTCTGAACGGATGTGT |
| 6 | 2 | TUB5-RV | CGAGATCTGGGAATTCAAGAAAAAATGCCAACG |
| 7 | 3 | TETAVR5-FW | CGATCCTAGGATGTCTAGATTAGATAAAAG |
| 8 | 4 | TETPST3-RV | CGTCTGCAGTTAAGACCCACTTTCACATTTAAG |
| 9 | 5 | T3 | ATTAACCCTCACTAAAGGGAA |
| 10 | 6 | SAG1/1634-RV | CGATAAGCTTTCGGGGGGGCAAGAATTGTGT |
| 11 | 7 | REV 13A | GCGCCCCATGGTGACGGAGAAAAATCG |
| 12 | 8 | REV 13B (nested | GGGAACCGCAAGGTGGGAGCGGAGAAC |
| | | primer) | |
| 13 | 9 | S13PROMFUS FW | GCATAAGCTTCCTCGCAGAGATTGTCAGTG |
| 14 | 10 | S13PROMFUS RV | GCATTCTAGAGGCAGACATGCCCTTTCCAGG |
| 15 | 11 | LACZ-AVRII FW | CGATCCTAGGATGACCATGATTACGGATTCACT |
| 16 | 12 | LACZ-PSTI RV | CGATCTGCAGTTATTTTTGACACCAGACCAA |
| 17 | 13 | S13INSTETO + 3FW | GGTTCTCCCCTCAATCCCTATCAGTGATAGAGATCTC |
| | | | TCTTCCTTTCTCT |
| 18 | 14 | S13INSTETO + 3RV | AGAGAAAGGAAGAGAGATCTCTATCACTGATAGGGAT |
| | | | TGAGGGGAGAACC |
| 19 | 15 | S13SUBTETO − 23FW | CTACGCGGCCGACGGTCCCTATCAGTGATAGAGATCT |
| | | | TCCTCGACGGGTTC |
| 20 | 16 | S13SUBTETO − 23RV | GAACCCGTCGAGGAAGATCTCTATCACTGATAGGGAC |
| | | | CGTCGGCCGCGTAG |
| 21 | | S13NOTI-FW | CGATGCGGCCGCGTCAGTGCATGACACAACCG |
| 22 | | S13SACI-RV | GCTAGAGCTCCTGTAAGTCGCCAGAGAAGCAC |
| 23 | | M13-REV | AACAGCTATGACCATGATTACGC |
| 24 | | S13CL FW3 | CGATAGTGTGCAATAACAGG |
| 25 | | HRCHECK II 5 S13- | GTCGAGTCCTGTAGGTTCATC |
| | | FW | |
| 26 | | HRCHECK II S13-RV | CTCCGAAGGAGTCTCTCAGTG |
| 27 | | T7 | AATACGACTCACTATAG |
| 28 | | HXGPRT/BGLII-FW | CGATAGATCTAAAATGGCGTCCAAACCCATTG |
| 29 | | HXGPRT/PSTI-RV | CGATCTGCAGTTACTTCTCGAACTTTTTGCG |

Construction of TubYFP/TR-sagCAT (9332 bp).

Plasmid ptubYFP/TR-sagCAT was engineered stepwise as described below. First the construct ptubCAT/GFP was made by amplifying the Toxoplasma gondii tubuline A (tub) promoter from the ptubYFP/YFP-sagCAT construct (Llopis, J. et al., PNAS 97(8): 4363-4368 (2000)) using the primers SAG3FW (#1, SEQ ID NO: 5) and TUB5RV (#2, SEQ ID No: 6). The PCR product as well as the plasmid pdhfrCAT/GFP (Striepen, B. et al., Molecular and Biochemical Parasitology 92: 325-338 (1998)) were digested with HindIII and BgIII, and ligated with each other. This resulted in ptubCAT/GFP where the dhfr promoter has been replaced by the tub promoter. The resulting plasmid is based on Bluescript pKS® (Stratagene, La Jolla, Calif.) and contains the α-tubuline promoter separated from the fusion of chloramphenicol acetyl transferase (CAT) coding sequence with green fluorescent protein coding sequence by a BgIII site.

To obtain the ptubYFP/TR construct the CAT coding sequence was exchanged for yellow fluorescent protein (YFP) and the GFP coding sequence was exchanged for tet-repressor coding sequence (tetR). The YFP gene was cut out of the ptubYFP/YFP-sagCAT construct by BgIII and AvrII, and ligated between BgIII and AvrII site of the ptubCAT/GFP construct replacing the CAT coding sequence. The tetR coding sequence was amplified by PCR from E. coli Tn10 (Hillen, W. and Berens, C., Annu. Rev. Microbiol. 48: 345-369 (1994)) using the primers TETAVR5-FW (#3, SEQ ID NO: 7) and TETPST3-RV (#4, SEQ ID NO: 8), digested by AvrII and PstI, and ligated in the construct by exchanging GFP coding sequence for the tetR coding sequence. The resulting plasmid was named ptubYFP/TR.

Finally a CAT selection cassette was inserted upstream of the tub promoter, resulting in the ptubYFP/TR-sagCAT plasmid. This was done by amplification of the CAT-cassette from the ptubYFP/YFP-sagCAT construct mentioned before using the primers T3 (#5, SEQ ID NO: 9) and SAG1/1634 RV (#6, SEQ ID NO: 10), digested with HindIII and ligated into teh unique HindIII site of the ptubYFP/TR construct.

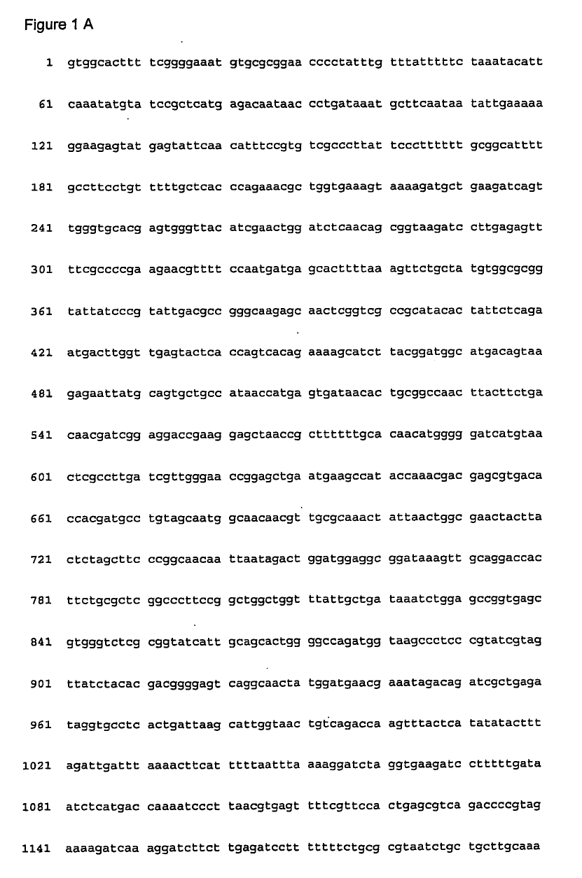

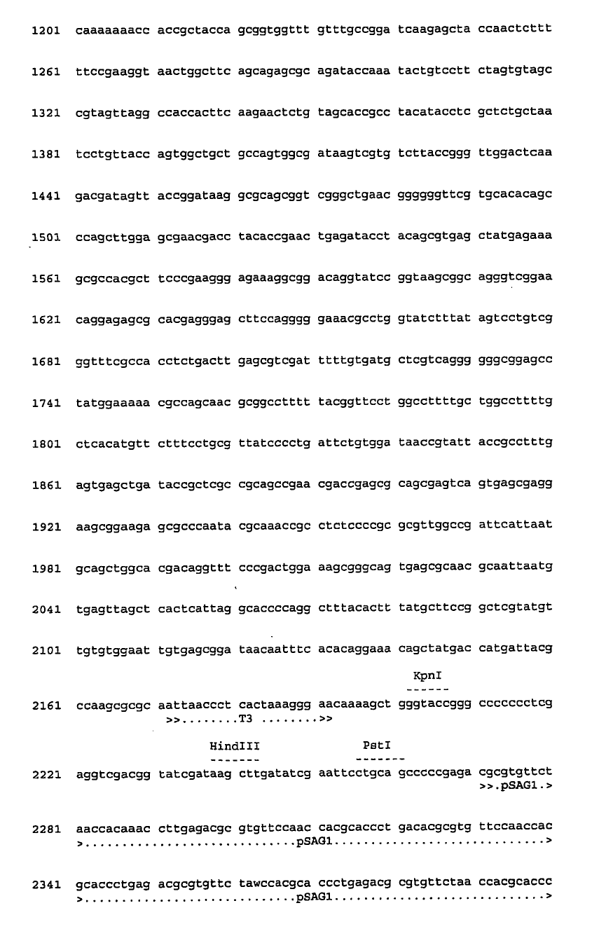

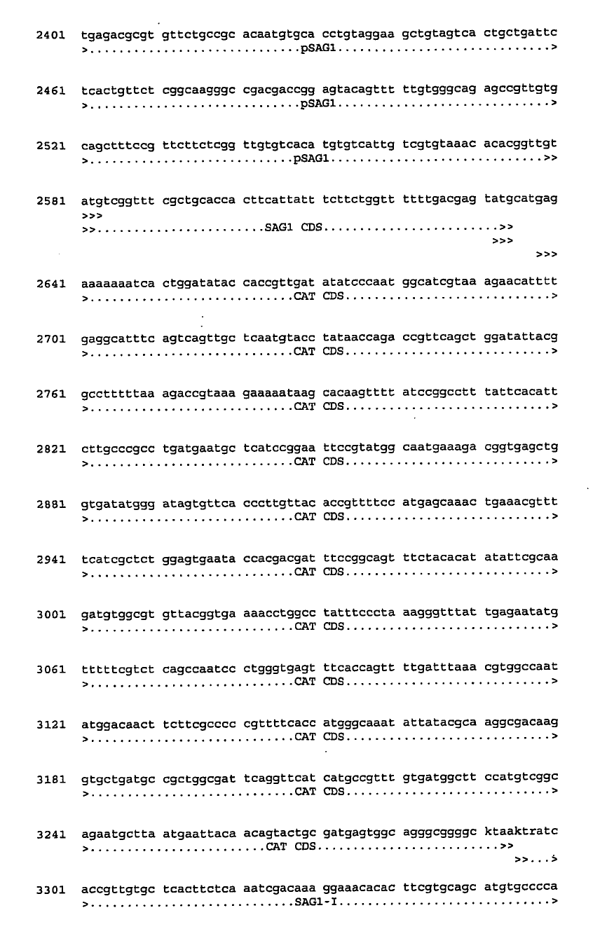

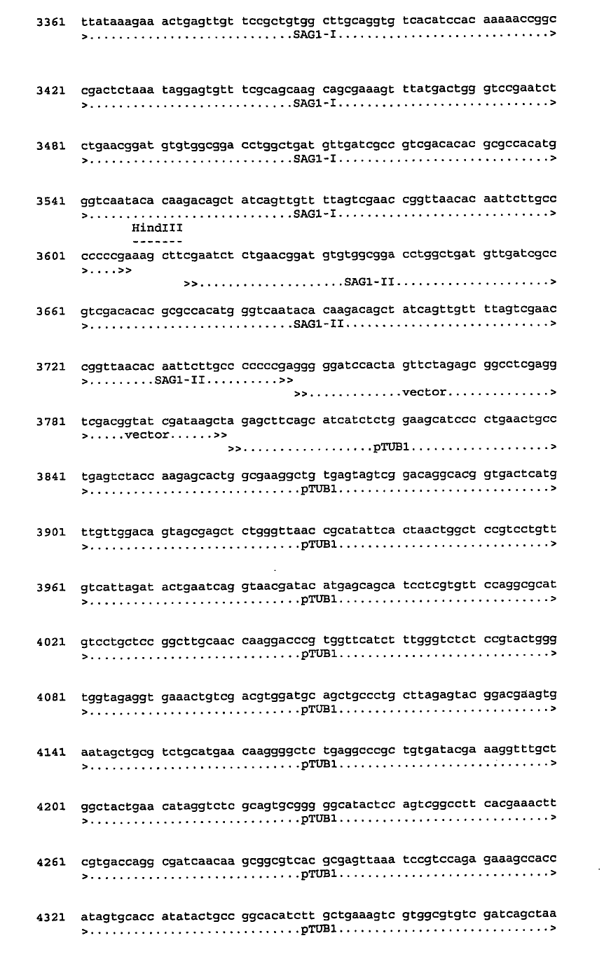

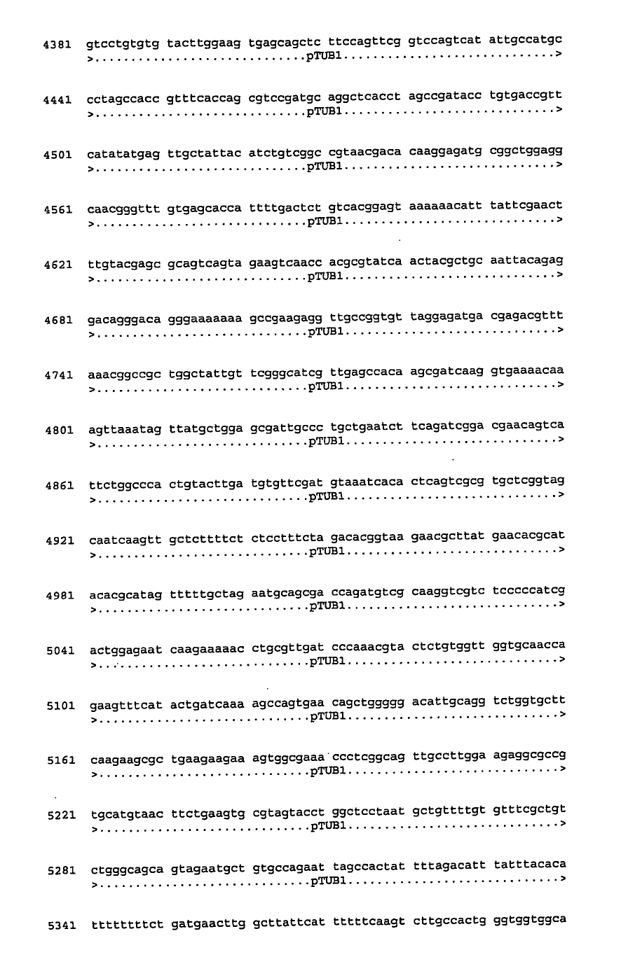

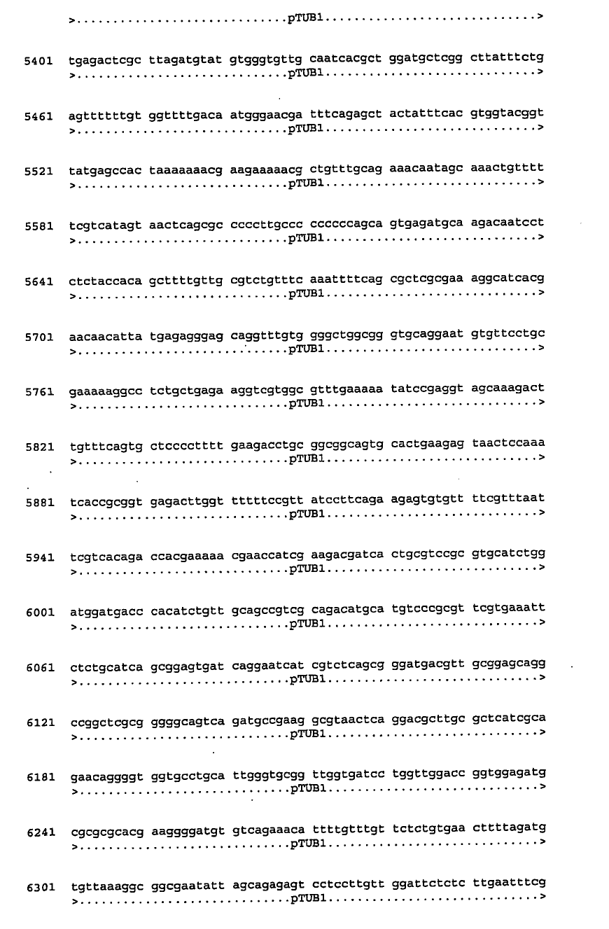

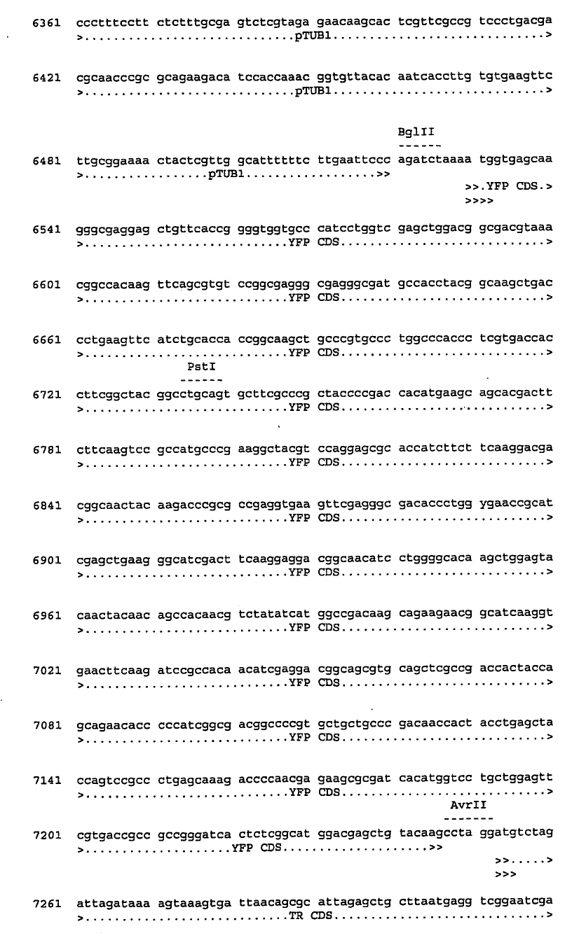

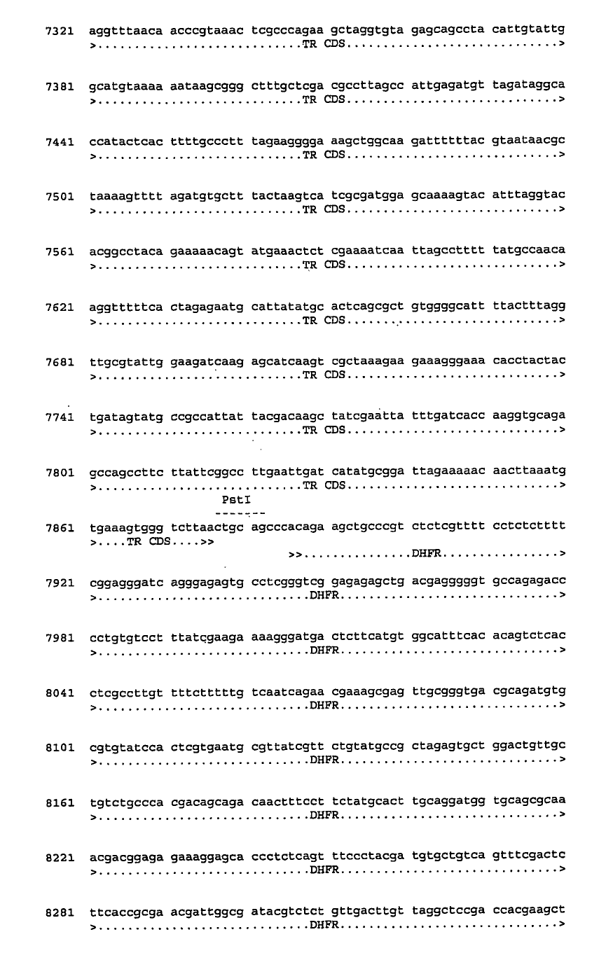

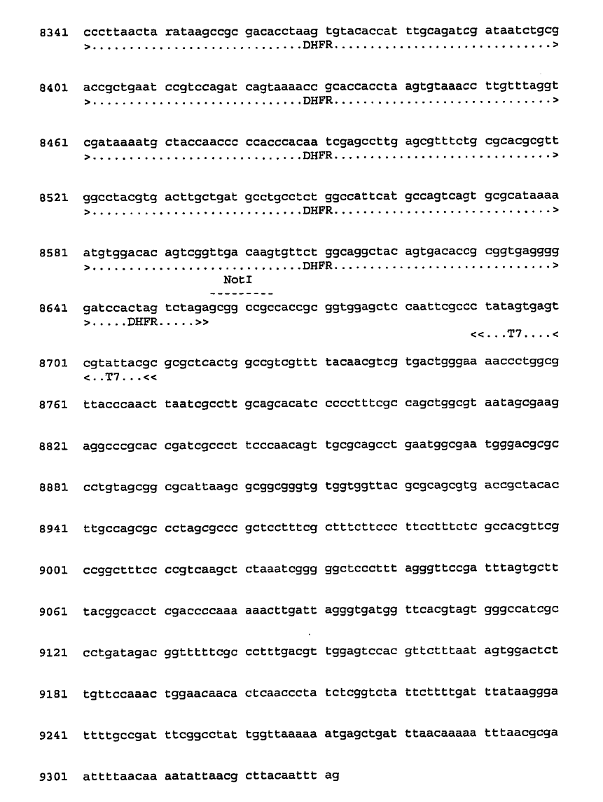

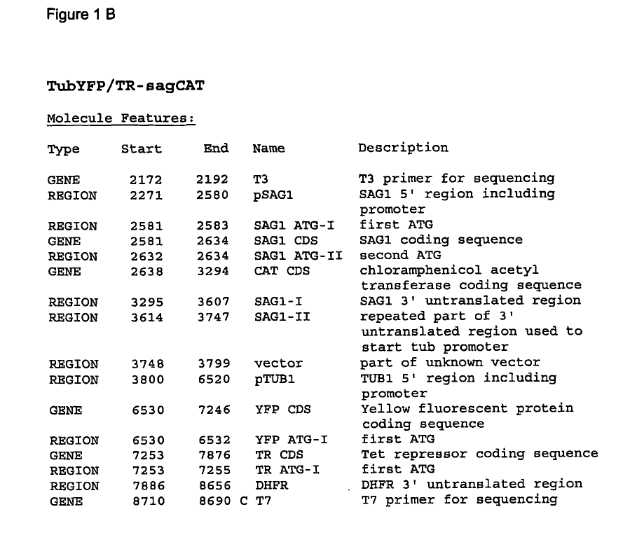

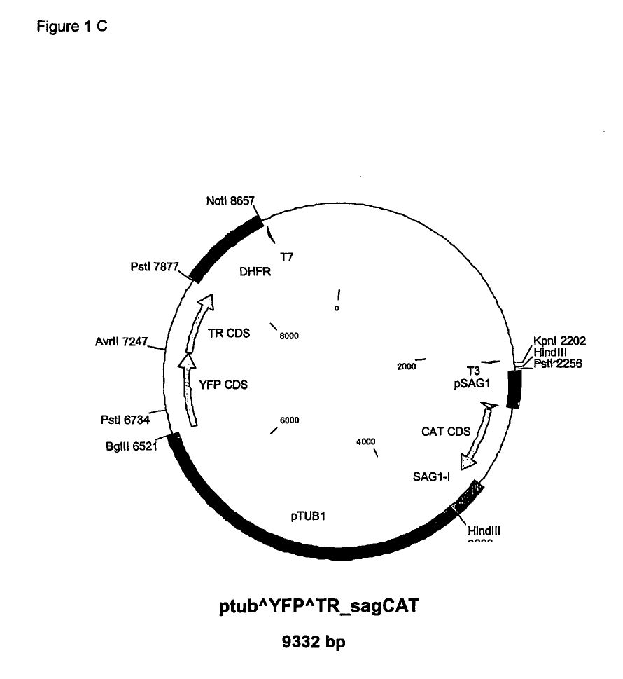

The construction of TubYFP/TR-sagCAT and its full sequence are presented in FIG. 1.

### Example 2

Determination of the Start Transcription Site of the Ribosomal Protein Gene S13 of Toxoplasma gondii

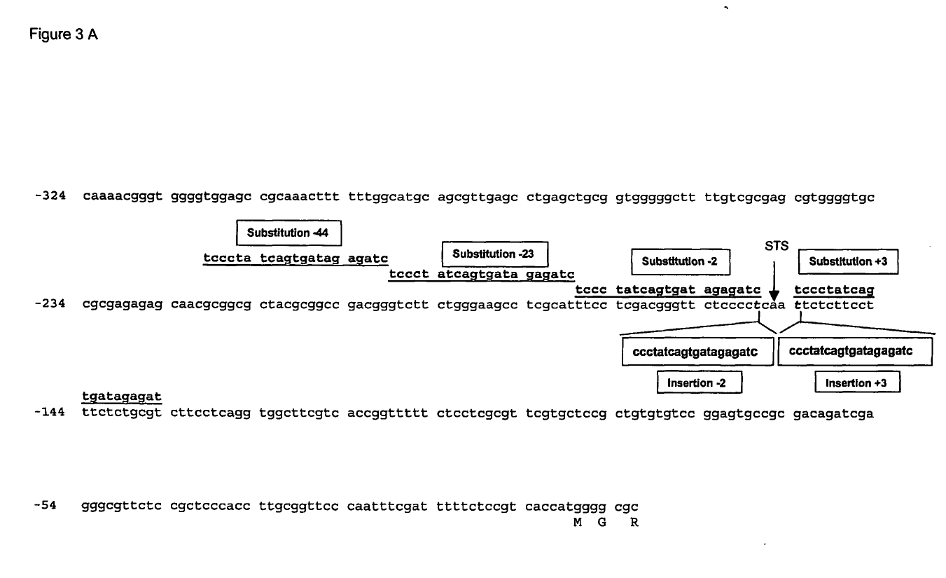

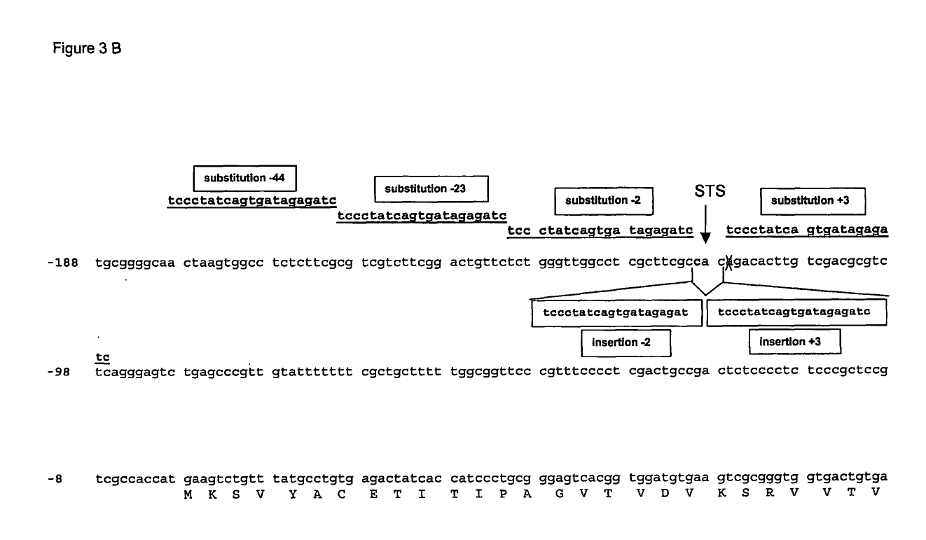

In order to determine the start of transcription of the ribosomal protein gene S13, RNA was isolated from Toxoplasma gondii RHΔHXGPRT tachyzoïtes grown in Vero cells. Using the GeneRacer® kit (Invitrogen) gene specific full-length cDNA was obtained from the total RNA. With this kit an RNA oligo was ligated to the ends of full-length mRNA. After reverse transcription by oligo dT had taken place, amplification by PCR with a GeneRacer primer binding to the RNA oligo together with a gene specific primer resulted in a product. Then the start of transcription (STS) could be determined. This was done for the ribosomal protein gene S13 using the following primers: REV13A (#7, SEQ ID NO: 11) and REV13B (#8, SEQ ID NO: 12). Primer #7 was used together with the GeneRacer primer to get a product after which primer #8 was used for the nested PCR. The PCR product showed three bands; two weak bands and a strong band. The band showing the highest amount of product has been isolated and the STS was determined and indicated as position 0. In FIG. 3 A and 3 B, the STS is also represented in relation to the startcodon.

### Example 3

S13/LZ Constructs

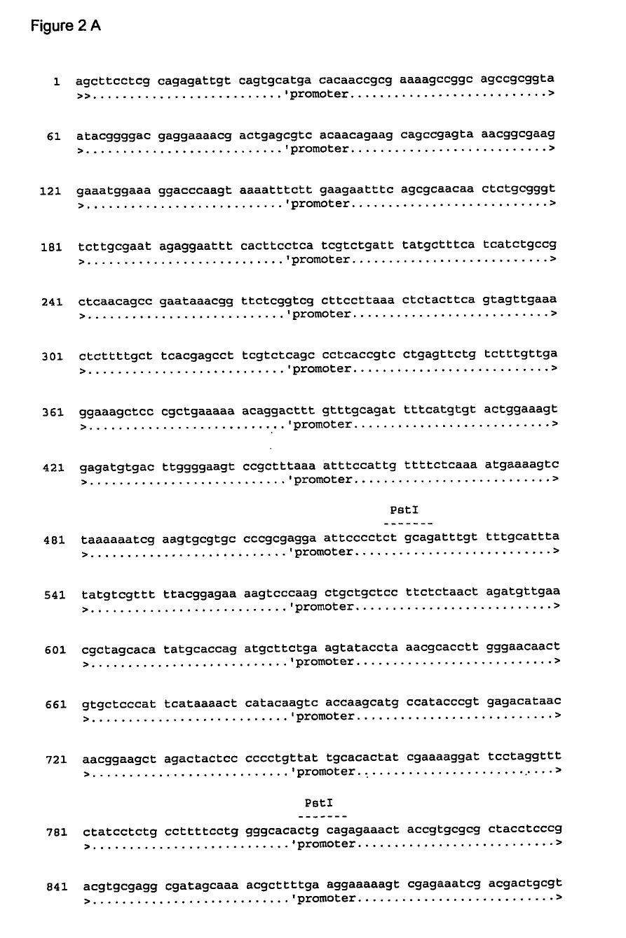

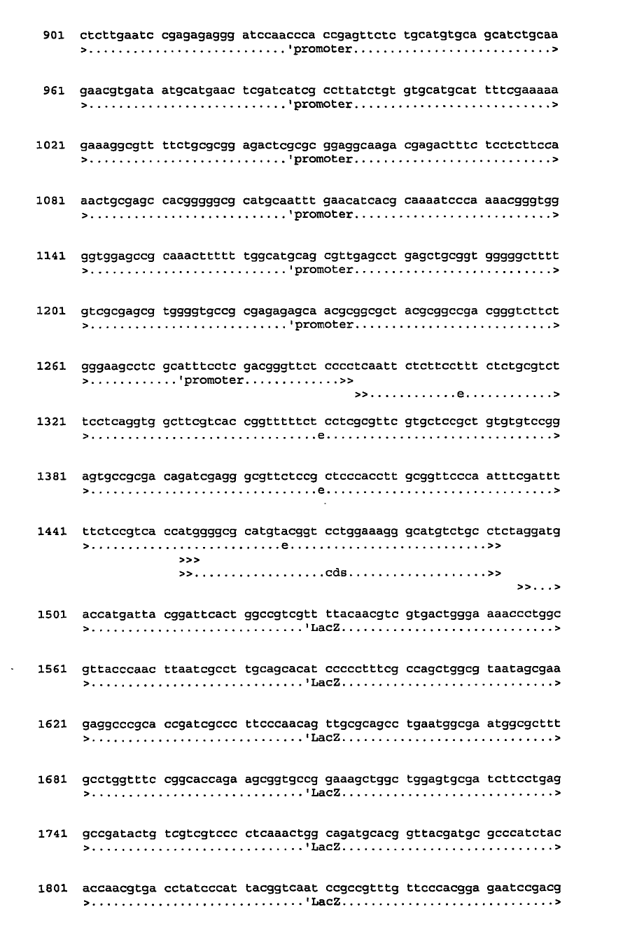

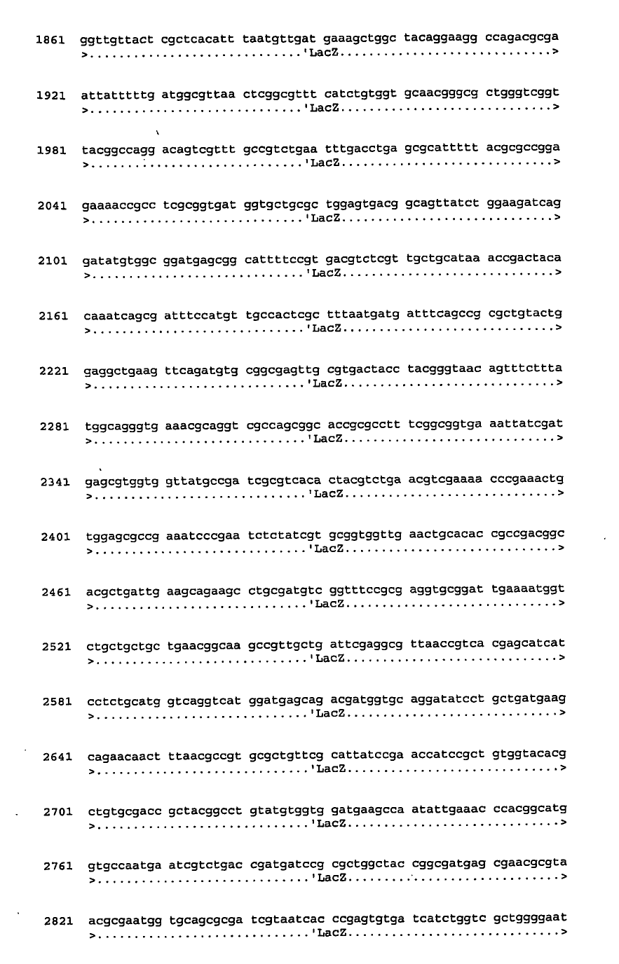

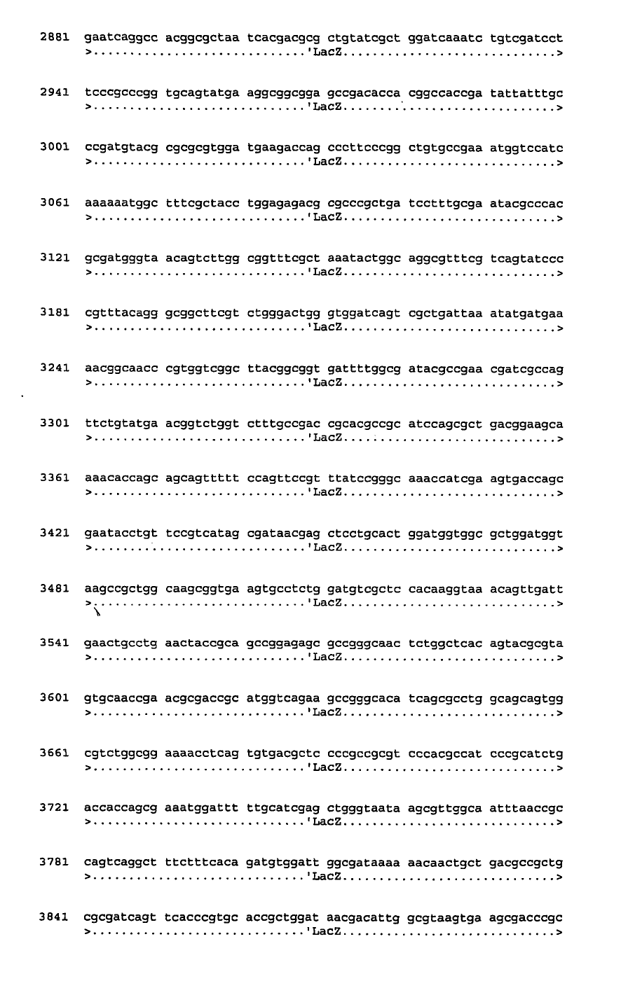

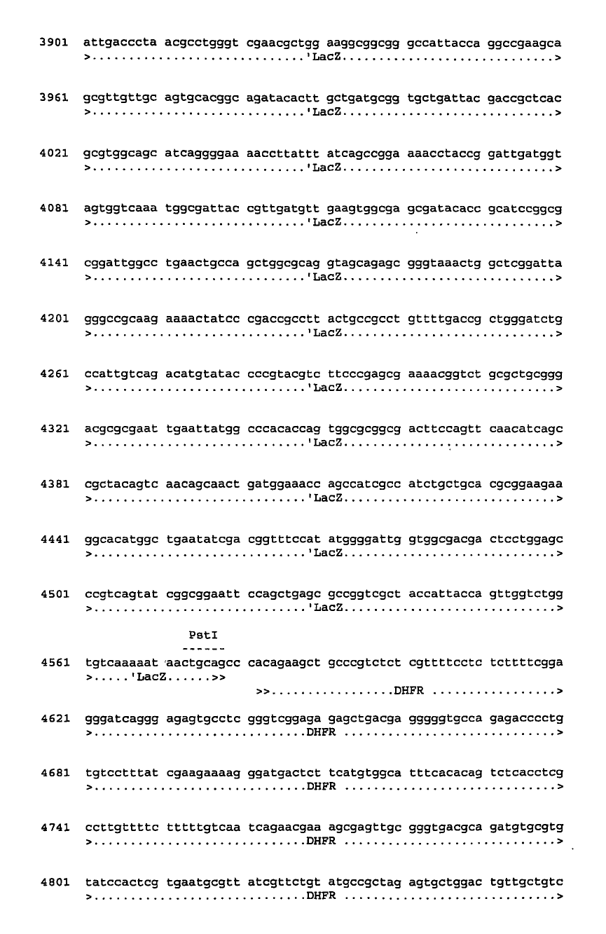

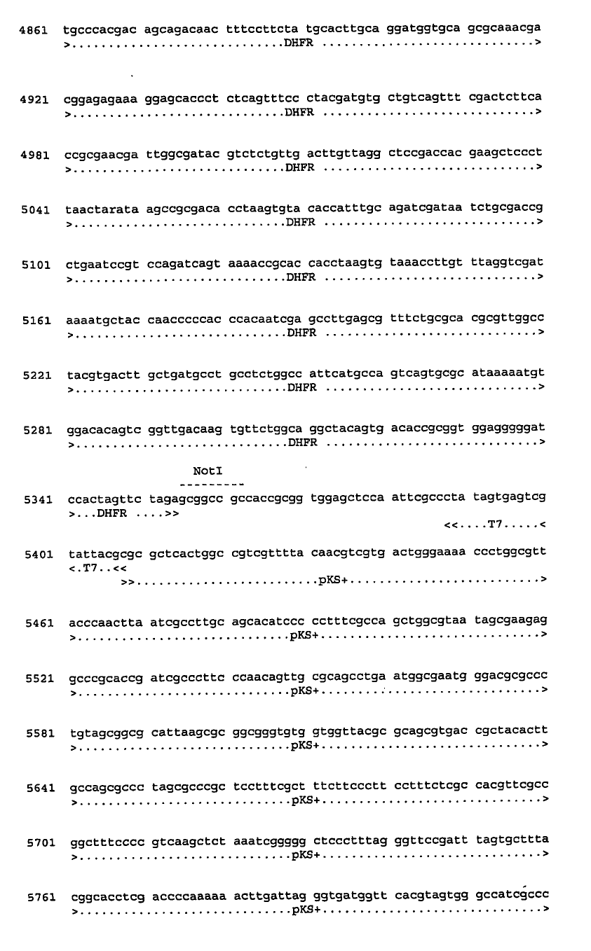

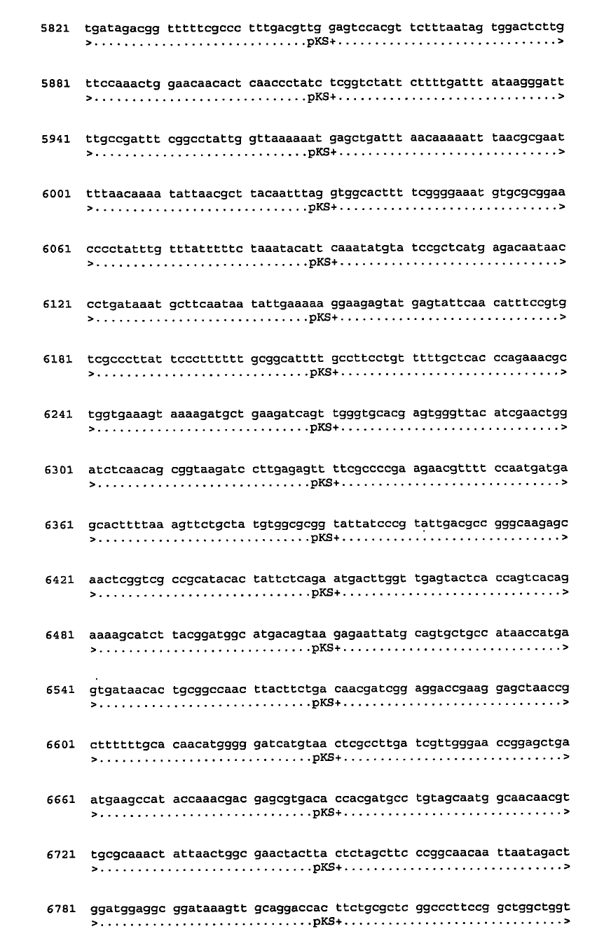

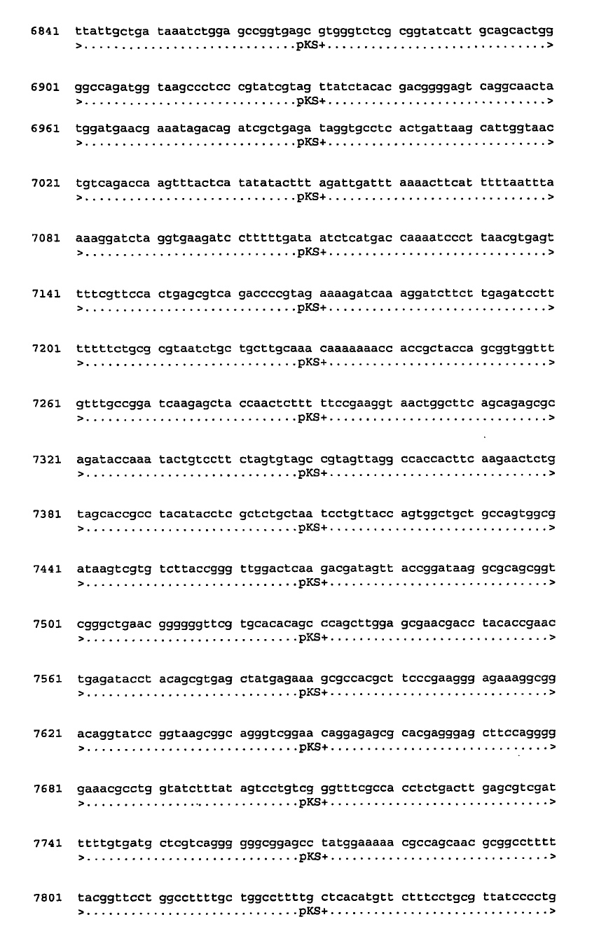

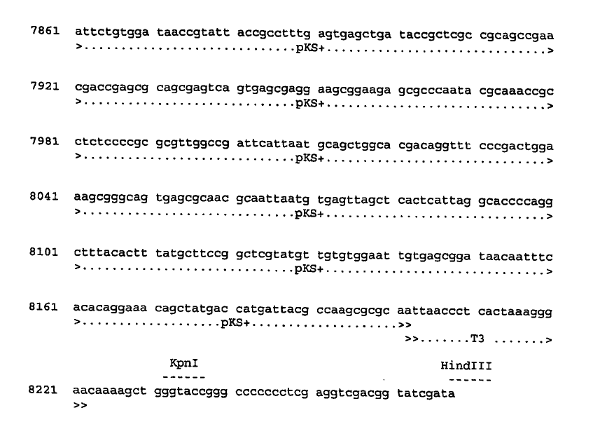

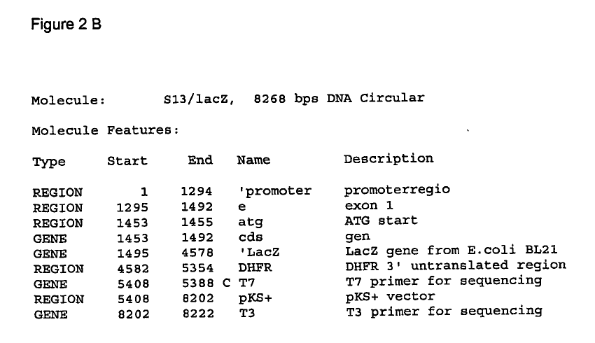

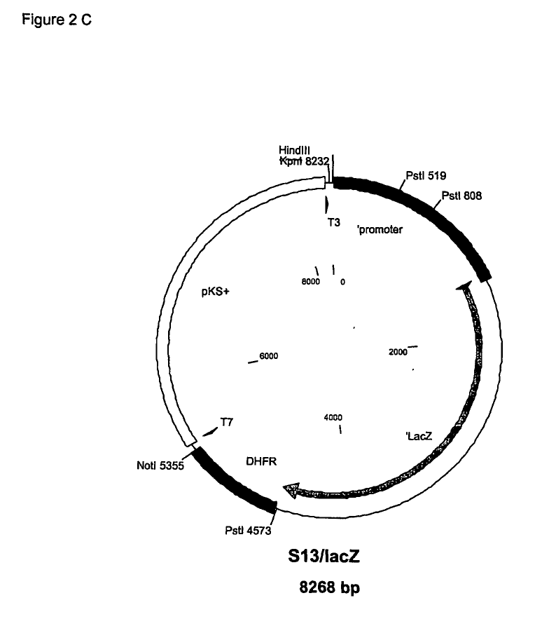

In order to test inducible expression by the tet repressor several reporter constructs were made with the lacZ gene under control of the S13 promoter with or without the presence of a single tetO site. First the plasmid S13/lacZ was made (see FIG. 2 for the structure and sequence of the final construct) and subsequently this plasmid was used to insert or substitute sequences for a tetO site as described below.

The promoter region of S13 was amplified by PCR from the genomic DNA of the Toxoplasma gondii RH/ΔHXGPRT strain with the primers S13PROMFUS FW (#9, SEQ ID NO: 13) and S13PROMFUS RV (#10, SEQ ID NO: 14). The lacZ coding sequence was amplified by PCR from the genomic DNA of BL21 bacteria with the primers LACZ-AVRII FW (#11, SEQ ID NO: 15) and LACZ-PSTI RV (#12, SEQ ID NO: 16). Subsequently the S13 PCR product was digested by HindIII and Xbal while the lacZ PCR product was digested by AvrII and PstI. The plasmid ptubYFP/YFP-sagCAT was used to exchange the ptubYFP part together with the CAT selection cassette for the S13 promoter part. The remaining YFP gene was exchanged for the lacZ gene, resulting in S13/lacZ plasmid. The S13/lacZ plasmid was used to insert or substitute sequences for a single tet operator (tetO) site

(5′-TCCCTATCAGTGATAGAGATC-3′) by site-directed mutagenesis. This was done using the QuickChange® site-directed mutagenesis kit (Stratagene). The tetO was inserted or substituted in the vicinity of the determined STS. The primers S13INSTETO+3 FW (#13, SEQ ID NO: 17) and S13INSTETO+3 RV (#14, SEQ ID NO: 18) were used to insert a tetO site at position +3 related to STS, which is indicated as 0. The primers S13SUBTETO−23 FW (#15, SEQ ID NO: 19) and S13SUBTETO−23 RV (#16, SEQ ID NO: 20) were used to substitute sequences for a tetO site between −43 and −23 related to STS. These two constructs, S13instetO+3/lacZ and S13subtetO−23/lacZ together with the S13/lacZ construct have been tested in the Toxoplasma gondii strains RHΔHXGPRT, REP½ (Meissner, M. et al., Nucleic Acids Research 29 (22), E115 (2001)) and tubYFP/TR by a CPRG assay (Seeber, F. et al., Gene 169: 39-45 (1996)) in the absence or presence of tetR and (anhydro)tetracycline.

The S13/lacZ construct is shown in FIG. 2 and the sites of substitution or insertion of the tet operator in the S13/lacZ construct are shown in FIG. 3A.

L9/LZ Constructs

tetO insertions/substitutions into the rp-L9 promoter are presented in FIG. 3B

### Example 4

Selection of stable transfectant Toxoplasma parasites carrying pTub-YFP-TR-sagCAT. Electroporation of Toxoplasma parasites was done as described by Roos, D. S. et al. (“Methods in Microbial Pathogenesis” In Methods in Cell Biology (1994), D. G. Russell, editor).

Selection of the stable transfectants was done according to Kim, K., et al. (Science 262(5135): 911-4 (1993)).

Electroporation of S13/LZ, S13i+3lacZ and S13s−23/lacZ constructs was again done according to Roos, D. S. et al. (1994, supra).

Results of Example 4

Determination of LacZ expression driven by an S13 promoter containing a single tet-operator, electroporated into the tub-YFP-TR strain.

The following constructs have been tested:
a) S13/LZ: This is the tub-YFP-TR transfectant Toxoplasma strain, transiently transfected with the LacZ gene under the control of the S13 ribosomal protein gene promoter. There is no tet-operator-site present in this construct.
b) S13i+3/lacZ: This is the tubYFP-TR transfectant Toxoplasma strain, transiently transfected with the LacZ gene under the control of the S13 ribosomal protein gene promoter, which additionally carries a tet-operator-site inserted at site +3 relative to the STS (see FIG. 3A).
c) S13s−23/lacZ: This is the tub-YFP-TR transfectant Toxoplasma strain, transiently transfected with the LacZ gene under the control of the S13 ribosomal protein gene promoter which additionally carries a tet-operator-site has been substituted at site −23 relative to the STS (see FIG. 3A).

Similar constructs of tetO insertions/substitutions into rp-L9 promoters are presented in FIG. 3B.

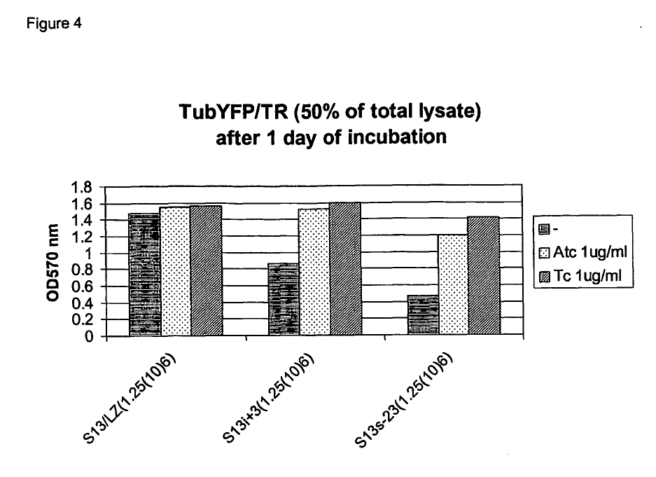

As can be seen in FIG. 4, tub-YFP-TR produces the same level of LacZ in both the presence and absence of anhydro-tetracycline and tetracycline, as expected.

Transfection with construct S13i+3/lacZ results in the production of an amount of LacZ in the absence of anhydro-tetracycline and tetracycline, that is half the amount of LacZ produced in the presence of anhydro-tetracycline and tetracycline. This clearly shows the inducibility of LacZ-transcription in this strain.

Transfection with construct S13s−23/lacZ results in the production of an amount of LacZ in the absence of anhydro-tetracycline and tetracycline, that is about ⅓ of the amount of LacZ produced in the presence of anhydro-tetracycline and tetracycline. This again clearly shows the inducibility of LacZ-transcription in this strain.

These results moreover prove that the site at which the tet-operator site is located relative to the STS, is not very critical. It additionally proves that the tet-operator site may be introduced by both insertion and substitution.

CPRG-assay of transient transfectants electroporated with a construct comprising a LacZ gene driven by an S13 promoter comprising a single tet-operator or a double tet-operator. In this assay the following constructs were compared:
a) S13/LZ as described above
b) S13s−23/lacZ(I) as described above (=S13s−23/lacZ)
c) S13s−23/lacZ(ll) which equals S13s−23/lacZ except for the fact that an additional tet-operator site has been cloned immediately downstream of the first tet-operator. The construct was assembled using similar techniques as for S13s−23/lacZ(I).

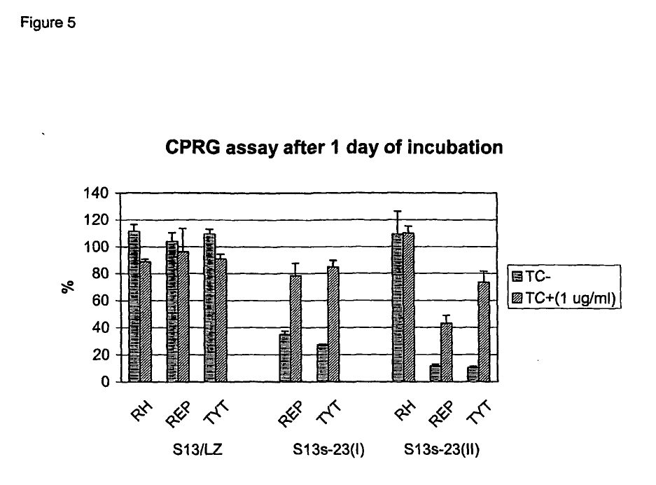

As follows from FIG. 5, both the synthetic tet-repressor gene (Meissner) mentioned above and a fusion tet-repressor gene (tub-YFP-TR) according to the invention are capable of blocking the transcription of LacZ in the absence of tetracycline. More strikingly, it clearly follows that the blocking of expression is between 3 and 4 times better when two adjacent tet-operator sites are used compared to the use of a single tet-operator.

CPRG-assay of transient transfectants comparing LacZ expression in a strain comprising the synthetic tet-repressor gene (Meissner) as described above, and a strain comprising a fusion tet-repressor gene according to the invention.

As follows surprisingly from FIG. 5, a fusion tet-repressor protein according to the invention gives a significantly better blocking of the transcription of LacZ when compared to the blocking found with synthetic tet-repressor protein (Meissner) as described above. Also, surprisingly, a much better induction of LacZ transcription is found with a fusion tet-repressor gene according to the invention when compared to the induction found with synthetic tet-repressor gene (Meissner) mentioned above.

### Example 5

Insertion of tet Operator Elements in the Ribosomal Protein S13 Locus using Homologous Recombination with the Hit-and-run Mutagenesis Procedure.

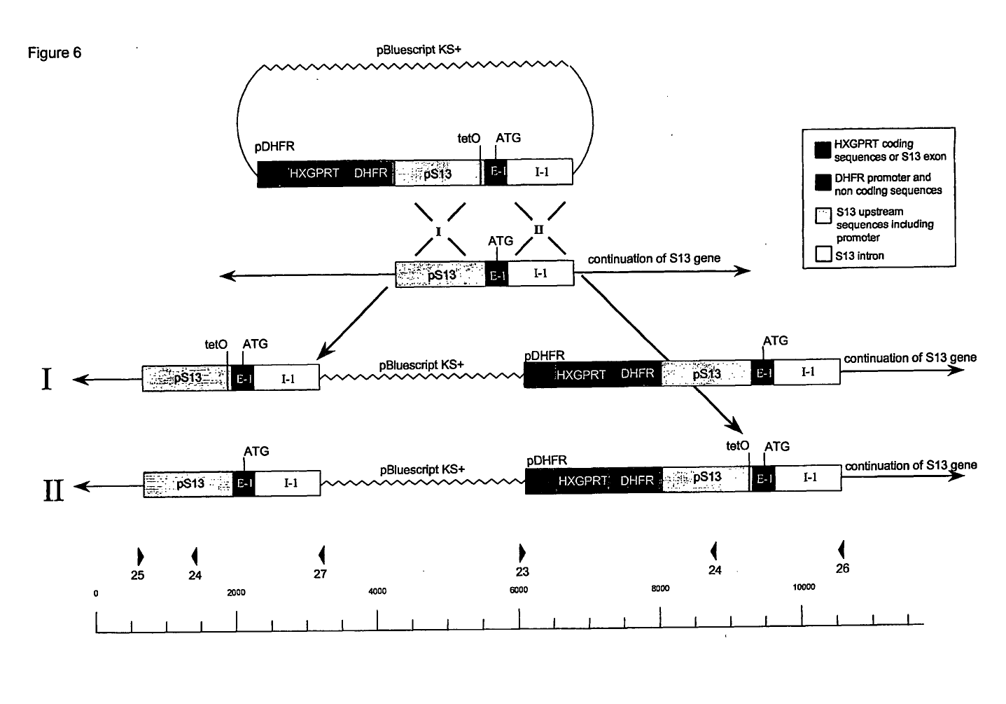

To integrate a tet operator site on the genome at a specific locus, in this case the ribosomal protein S13 locus (S13), homologous recombination is required. For homologous recombination a large sequence part (in this case ˜1200 bp) upstream and downstream of the integration site is needed to obtain a homologous recombination instead of a non-homologous recombination. As described by Donald et al. (Mol. Biochem. Paras. 91: 295-305 (1998)), it is possible to integrate a sequence element at a specific locus in two steps using the hypoxanthine-xanthine-guanine phosphoribosyltransferase (HXGPRT) gene as a selection marker. In detail, a transfected plasmid containing part of the S13 locus near the integration site, which is preceded by an HXGPRT cassette will homologously recombine once with the homologous genomic DNA S13 locus, creating a pseudodiploid type I or II (FIG. 6). This is performed under positive selection for HXGPRT by mycophenolic acid and xanthine as described (Donald et al. 1998, supra). Subsequently the second homologous recombination is selected with 6-thioxanthine against HXGPRT which results in loss of the pseudodiploid and creation of a tachyzoite with or without a tet operator site integrated at the S13 locus (˜1:1 ratio). This procedure is called hit-and-run mutagenesis. To perform this procedure first a plasmid was made containing an HXGPRT selection cassette under the control of a DHFR promoter. RNA was isolated from Toxoplasma gondii RH tachyzoites. This RNA was used for making cDNA using SUPERSCRIPT™II RnaseH-Reverse Transcriptase (Gibco BRL) and standard molecular biological procedures (Sambrook & Russell: “Molecular cloning: a laboratory manual” (2001), Cold Spring Harbor Laboratory Press; ISBN: 0879695773). The HXGPRT coding sequence was amplified from the T. gondii RH tachyzoites' cDNA using primers HXGPRT/BGLII-FW (SEQ ID NO: 28) and HXGPRT/PSTI-RV (SEQ ID NO: 29) and splice variant-I was selected for further use (Donald et al., J. Biol. Chem. 271: 14010-14019 (1996)). Both the PCR product and plasmid pdhfrCAT/GFP (Striepen, B. et al., Mol. Biochem. Paras. 92: 325-338 (1998)) were digested with BgIII and PstI after which the CAT/GFP coding sequence was exchanged for the HXGPRT coding sequence, resulting in a dhfrHXGPRT construct named pminiHXGPRT.

Subsequently a DNA part containing the area both upstream (˜1200 bp) and downstream (˜1200 bp) of the tet operator integration site (−43/−23 relative to STS) was PCR amplified from genomic DNA of T. gondii RH tachyzoites using primers S13NOTI-FW (SEQ ID NO: 21) and S13SACI-RV (SEQ ID NO: 22). Both this PCR product and the pminiHXGPRT were digested by Notl and Sacl after which the PCR product was ligated downstream of the HXGPRT cassette. Finally, a tet operator was inserted with primer S13SUBTETO-23FW (SEQ ID NO: 19) and primer S13SUBTETO−23RV (SEQ ID NO: 20) by substitution using site-directed mutagenesis as described in Example 3, creating pS13s−23/pminiHXGPRT.

Circular pS13s−23/pminiHXGPRT plasmid was electroporated as described previously (Example 4) into RHΔHXGPRT tachyzoites. After infection into Vero cell monolayers, mycophenolic acid/xanthine selection was started as described by Donald et al. (1998, supra).

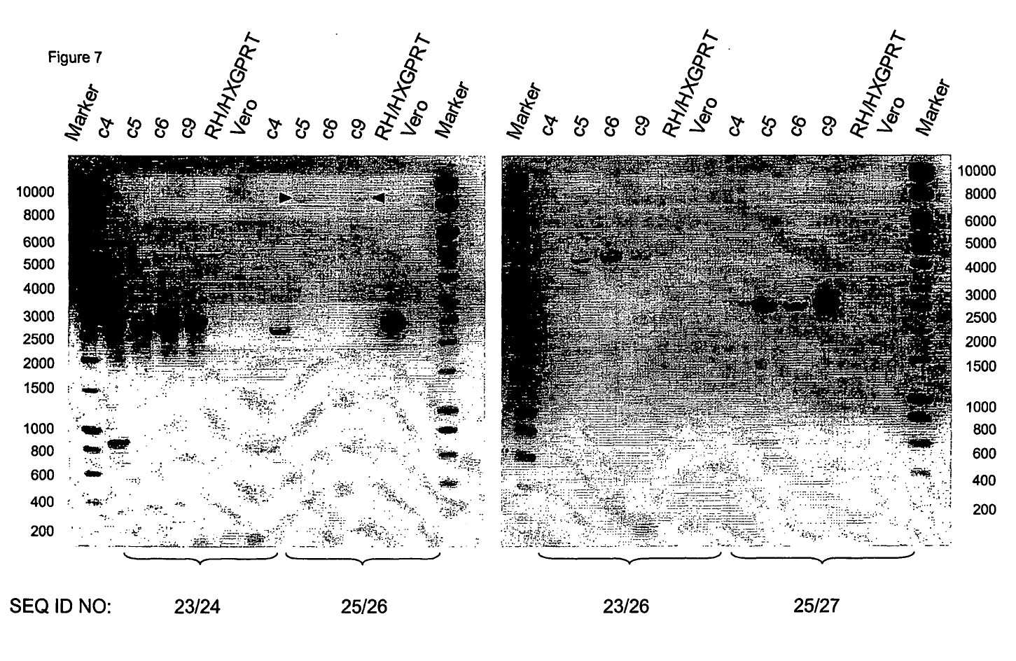

After stable transfectants were generated according to Kim, K., et al. (Science 262 (5135): 911-4 (1993)), several clonal parasite lines were picked. Genomic DNA was isolated from each of these clones. PCR analysis was performed on these genomic DNA samples to check for the presence or absence of the pseudodiploid form in these transfectants using the primers M13-REV (SEQ ID NO:23), S13CL FW3 (SEQ ID NO: 24), HRCHECK II 5 S13-FW (SEQ ID NO: 25), HRCHECK II S13-RV (SEQ ID NO: 26), and T7 (SEQ ID NO: 27). Four clones (c4, c5, c6 and c9) were analysed in detail and the genomic DNA of the strain RHΔHXGPRT and of Vero cells was used as a negative control. Different primer combinations (FIG. 6) were used to amplify by PCR the genomic DNA of these samples, these are listed as: 23/24, 25/26, 23/26, and 25/27, meaning the combination of primers of SEQ ID NO: 23 and 24, etc. Results are presented in FIG. 7. Primer combination 23/24 shows the presence of the plasmid in the different clones. Primer M13-REV (SEQ ID NO: 23) anneals to the vector part which is absent in untransfected parasites (RHΔHXGPRT). All transfected clones show bands of the correct size (2.8 kb), indicating that all stable transfectants have taken up the plasmid after electroporation and kept it during selection. Subsequently, the primer combination 25/26 shows whether the pseudodiploid form is present in the clones. On the genome, both primers are located upstream (primer HRCHECK II 5 S13-FW (SEQ ID NO: 25)) or downstream (primer HRCHECK II S13-RV (SEQ ID NO: 26)) of the S13 part present in the vector. If the pseudodiploid is not present, the “wild type” S13 situation will be PCR amplified which results in a product of ˜2.6 kb as can be observed with clone c4 and the wild type parasite RHΔHXGPRT. This shows that clone c4 is a stable transfectant without a pseudodiploid, suggesting that non-homologous recombination occurred. The absence of the 2.6 kb PCR product for the clones c5, c6 and c9 indicates that these clones do contain the pseudodiploid form. In addition, a product of nearly 10 kb can be observed for clones c5 and c9, which is as expected when the pseudodiploid is present. No 10 kb product was detectable for clone c6. The PCRs with primer combination 23/26 and primer combination 25/27 were performed to demonstrate that the p13s−23/pminiHXGPRT vector is juxtaposed at both sides by the S13 locus. Primer M13-REV (SEQ ID NO: 23) is located in the vector sequence and primer HRCHECK II S13-RV (SEQ ID NO: 26) is located on the genome downstream of the homologous S13 part of the vector. Primer T7 (SEQ ID NO: 27) is located in the vector sequence and primer HRCHECK II 5 S13-FW (SEQ ID NO: 25) is located on the genome upstream of the homologous S13 part of the vector. In the wild type situation the primer combination 23/27 does not anneal to DNA and for that reason no PCR product can be amplified. In case of a pseudodiploid the primer combination 23/26 results in a product of 4.6 kb and the combination 25/27 results in a PCR product of 2.6 kb. The data presented in FIG. 7 demonstrate that indeed the positive clones show the right bands for both combinations whereas for the negative samples no products were observed.

This PCR analysis is therefore used to verify that the homologous recombination into e.g. the S13 locus by the hit-and-run mutagenesis procedure is performed successfully.

Legend to the Figures.

FIG. 1: Description of the TubYFP/TR-sagCAT construct:

FIG 1A: Full sequence: relevant regions are indicated below the sequence; restriction enzyme recognition sites are indicated above the sequence,

FIG. 1B: List of relevant features and regions of the TubYFP/TR-sagCAT construct,

FIG. 1C: Graphical map of the TubYFP/TR-sagCAT construct.

FIG. 2: Description of the S13/lacZ construct:

FIG. 1A: Full sequence: relevant regions are indicated below the sequence; restriction enzyme recognition sites are indicated above the sequence,

FIG. 1B: List of relevant features and regions of the S13/lacZ construct,

FIG. 1C: Graphical map of the S13/lacZ construct.

FIG. 3:

FIG. 3A: tetO insertions/substitutions in rp-S13 promoter:

Sequence of part of the ribosomal protein S13-promoter, also indicating the site of the +3 insertion and the −23 substitution, relative to the STS. Also indicated are the first three amino acids of the coding region.

FIG. 3B: tetO insertions/substitutions in rp-L9 promoter:

FIG. 4: Determination of the level of LacZ expression by tubYFP/TR stable transfectants electroporated with the constructs S13/LZ, S13i+3/lacZ and S13s−23/lacZ without antibiotics, in the presence of 1 μg/ml anhydro-tetracycline (Atc) or in the presence of 1 μg/ml tetracycline (Tc). The OD is an indication for the level of LacZ expression. The labels of the horizontal axis indicate that 1.25×10⁶ tachyzoïtes were used (50% of originally made amount).

FIG. 5: Determination of the LacZ expression level in different strains (RH, REP, tubYFP/TR) electroporated with the constructs S13/LZ, S13s−23/lacZ(I) and S13s−23/lacZ (II) RH represents the strain without tet-repressor gene. REP represents the strain carrying the synthetic tet-repressor gene (Meissner). TYT represents the strain carrying the fusion tet-repressor gene (tub-YFP-TR). Equal amounts of cells have been used in these comparative experiments. Experiments have been done in the absence or presence of tetracycline as indicated in the figure.

FIG. 6: Formation of type I and II pseudodiploid forms after first step of hit-and-run mutagenesis:

FIG. 7: PCR on genomic DNA of different clones to determine presence of pseudodiploid forms

1-20. (canceled)

21. An attenuated live parasite of the phylum Apicomplexa or the family of Trypanosomatidae, wherein said parasite comprises a ribosomal protein gene under the control of an inducible promoter.

22. The attenuated live parasite according to claim 21, wherein said parasite belongs to the Coccidia, the Piroplasmida or the Haemosporida.

23. The attenuated live parasite according to claim 22, wherein said parasite belongs to the family of the Eimeridiidae, Cryptosporidiidae or Sarcocystidae.

24. The attenuated live parasite according to claim 23, wherein said parasite belongs to the genus Eimeria, Cryptosporidium, Toxoplasma, Sarcocystis or Neospora.

25. The attenuated live parasite according to claim 22, wherein said parasite belongs to the family of the Babesiidae or the Theileriidae.

26. The attenuated live parasite according to claim 25, wherein said parasite belongs to the genus Babesia or Theileria.

27. The attenuated live parasite according to claim 22, wherein said parasite belongs to the genus Plasmodium.

28. The attenuated live parasite according to claim 21, wherein said parasite belongs to the genus Trypanosoma or the genus Leishmania

29. The attenuated live parasite according to claim 21, wherein said inducible promoter is based upon an operator site and a repressor protein capable of reversibly binding said operator site.

30. The attenuated live parasite according to claim 21, wherein said inducible promoter is inducible by antibiotics.

31. The attenuated live parasite according to claim 20, wherein said inducible promoter is inducible by tetracycline or anhydrotetracyclin, or a derivative thereof.

32. The attenuated live parasite according to claim 21, wherein a tetR-system is used as the inducible promoter.

33. The attenuated live parasite according to claim 1, wherein said ribosomal protein gene is the gene encoding L9, S3, plastid-S9 or S13, preferably L9, S3, plastid-S9 or S13 of Toxoplasma gondii.

34. A vaccine for combating parasitic infection comprising the attenuated live parasite of claim 21 and a pharmaceutically acceptable carrier.

35. A method for the production of the vaccine according to claim 34, said method comprising the mixing of a live attenuated parasite according to claims 1 and a pharmaceutically acceptable carrier.

36. A DNA-fragment encoding a tet-repressor fusion protein comprising the tet-repressor protein and a heterologous protein or a part thereof, said heterologous protein or a part thereof being fused to the N-terminal side of the tet-repressor protein, the monomeric form of said fusion protein having a molecular weight of less than 60 kD and being free of GPI-anchors, secretion/excretion signals and trans-membrane regions.

37. An attenuated live parasite according to claim 1, wherein said parasite comprises the tet-operator site and a DNA fragment encoding a tet-repressor fusion protein according to claim 36.

38. An aAttenuated live parasite according to claim 37, wherein said parasite comprises two or more tet-operator sites.
